(19) United States
(12) Patent Application Publication
(10) Pub. No.: US 2010/0273203 A1
(43) Pub. Date: Oct. 28, 2010

(54) METHODS AND COMPOSITIONS FOR DETECTING METABOLITES

(75) Inventors: Grover P. Miller, Little Rock, AR (US); Anna Radominska-Pandya, Little Rock, AR (US); Jeffery H. Moran, Little Rock, AR (US)

Correspondence Address: POLSINELLI SHUGHART PC, 700 W. 47TH STREET, SUITE 1000, KANSAS CITY, MO, 64112-1802, US

(73) Assignee: Board of Trustees of the University of Arkansas, Little Rock, AR (US)

(21) Appl. No.: 12/766,635

(22) Filed: Apr. 23, 2010

Related U.S. Application Data
(60) Provisional application No. 61/172,121, filed on Apr. 23, 2009.
(60) Provisional application No. 61/172,578, filed on Apr. 24, 2009.
(60) Provisional application No. 61/173,085, filed on Apr. 27, 2009.

(51) Int. Cl. C12Q 1/02; B01D 59/44

(52) U.S. Cl. 435/29; 250/282

(57) ABSTRACT

The present invention provides a metabolic profile, a database comprising a metabolic profile, a method for determining a metabolic profile, uses for a metabolic profile, and warfarin metabolites.

### CROSS REFERENCE TO RELATED APPLICATIONS

This application claims the priority of U.S. provisional application No. 61/172,121, filed Apr. 23, 2009, U.S. provisional application No. 61/172,578, filed Apr. 24, 2009, and U.S. provisional application No. 61/173,085, filed Apr. 27, 2009,each of which is hereby incorporated by reference in its entirety.

### GOVERNMENTAL RIGHTS

This invention was made with government support under contract number 200-2007-21729 and U90/CCU616974-07awarded by the Centers for Disease Control. The government has certain rights in the invention.

### FIELD OF THE INVENTION

The invention encompasses methods of determining metabolic profiles of a compound, uses for a metabolic profile, and metabolic compounds.

### BACKGROUND OF THE INVENTION

Activity of pharmaceutically active compounds is modulated, in part, by their metabolic inactivation and elimination. As a result, understanding the metabolic pathways associated with the inactivation and elimination of a compound provides valuable information about the pharmacokinetics of the compound. For instance, every year, two million people begin Coumadin (R-, S-Warfarin [War]) therapy in the United States. War is a challenging drug to accurately dose, both initially and for maintenance, because of its narrow therapeutic range, wide inter-patient variability, and long list of factors that can influence dosing. Recently, War was reported to be the fourth leading cause of adverse drug events. Therefore, the development of improved treatment and effective management in anticoagulant therapy strategies remains an important challenge. It is well established that effective personalization of War therapy may have to rely on much more than just pharmacogenomic information. The current challenge for personalizing War therapy is to develop better clinical diagnostics that can account for all confounding factors and develop better anticoagulant drugs by improving the understanding of War metabolic pathways. The current ability to personalize War therapy is limited because traditional approaches are much more complicated than previously appreciated and current pharmacogenomic approaches, which utilizes genetic information related to polymorphisms of cytochrome P450 (CYP)2C9 and VKORC1, are not answering all the questions. Underlying genetic factors account for only ˜35-40% of variations in patient response to War, which leaves significant room for improvement.

An alternative to genotyping is the strategy of metabolic phenotyping. This strategy utilizes a metabolomics approach to develop a metabolic profile for a subject. The profile may then be correlated with dose response, to provide better dosing efficacy and safety and/or may help identify better drug candidates.

### SUMMARY OF THE INVENTION

One aspect of the invention encompasses a warfarin metabolic profile of a subject. Generally speaking, the profile comprises the identity and stereochemistry of phase II warfarin metabolites in a sample from a subject. Typically, the profile is stored in a computer readable form.

Another aspect of the invention encompasses a method for determining a warfarin metabolic profile of a subject. Usually, the method comprises determining the identity and stereochemistry of phase II warfarin metabolites in a sample from the subject.

Yet another aspect of the invention encompasses a method for determining an effective dose range of warfarin for a subject. The method generally comprises determining the warfarin metabolic profile of the subject. Then, the metabolic profile is compared to a database that comprises at least one warfarin metabolic profile correlated to a known effective warfarin dose. A database profile similar to the subject's warfarin metabolic profile is selected, and the effective dose range of warfarin for the subject is the known effective warfarin dose correlated to the database profile.

Other aspects and iterations of the invention are described more thoroughly below.

### REFERENCE TO COLOR FIGURES

The application file contains at least one photograph executed in color. Copies of this patent application publication with color photographs will be provided by the Office upon request and payment of the necessary fee.

### BRIEF DESCRIPTION OF THE FIGURES

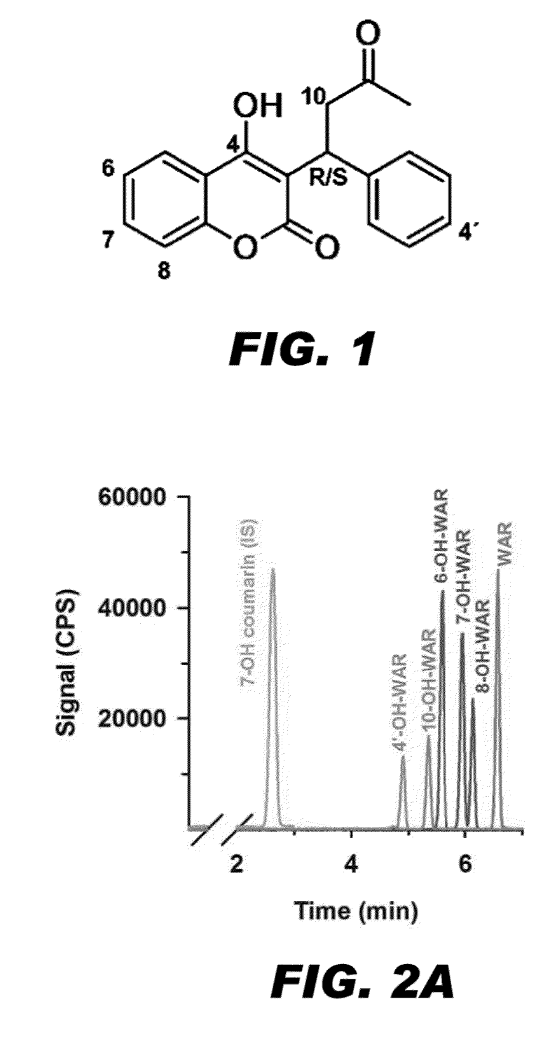

FIG. 1. Schematic representation of warfarin. Numbers indicate active sites for cytochrome P450 hydroxylations and subsequent UGT catalyzed conjugation.

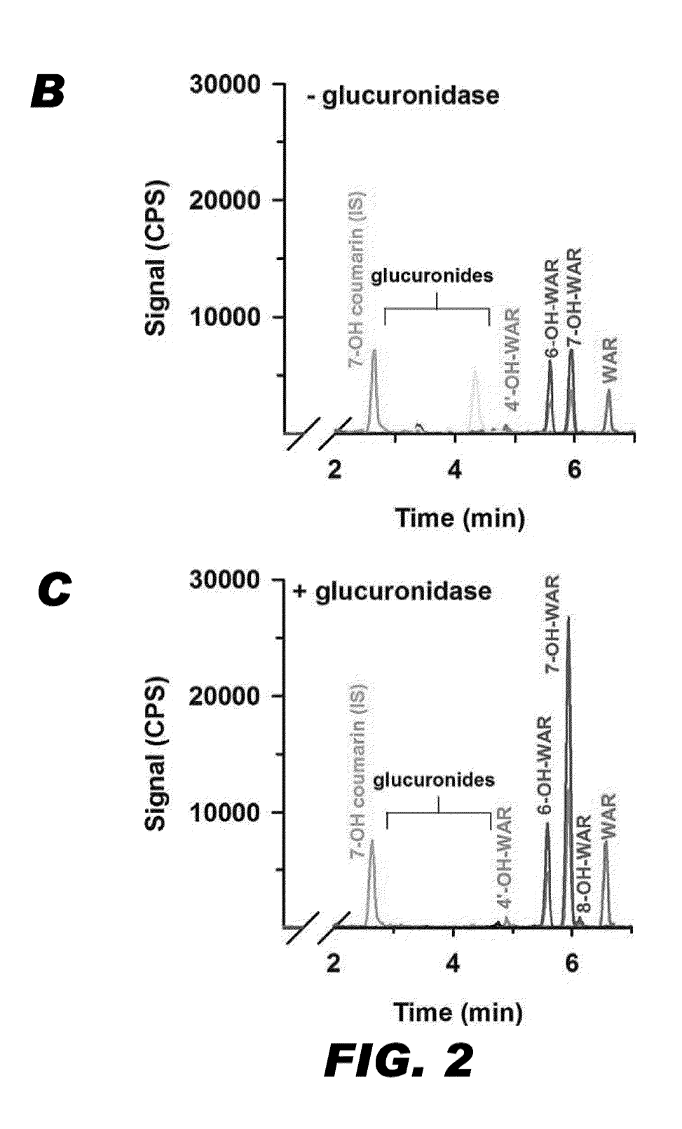

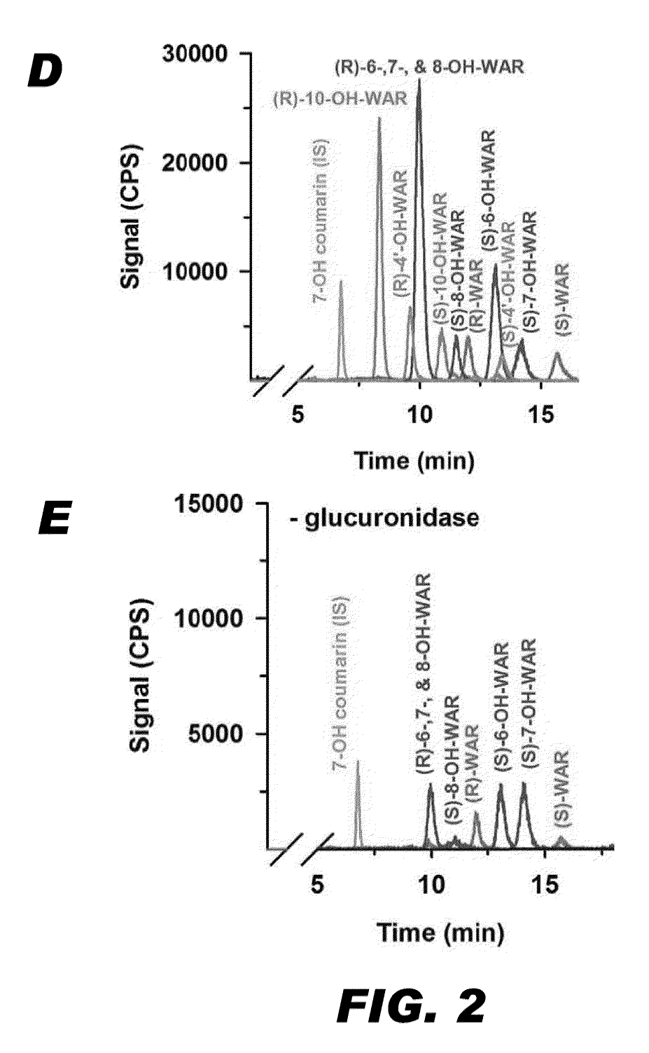

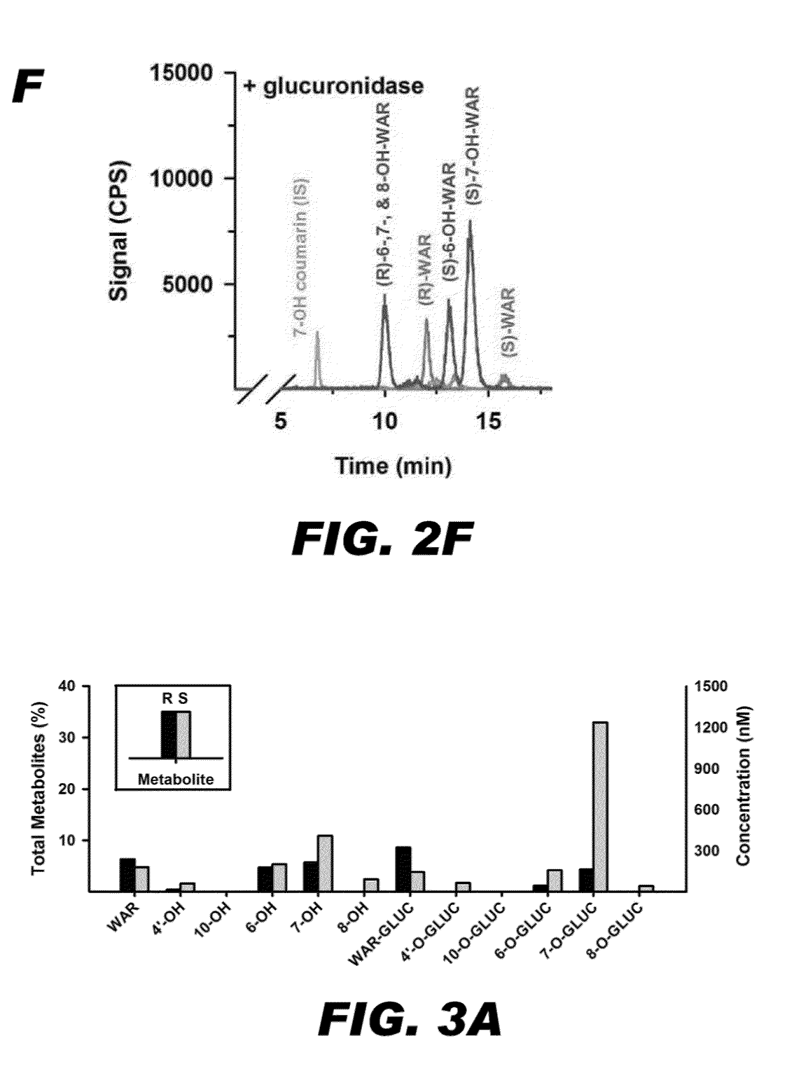

FIG. 2. Representative chromatograms produced during MRM experiments. (A-C) Represent the first analytical phase followed by (D-F) the second chiral analytical phase. Panels A and D represent 100 nm and 50 nM authentic standards, respectively. Panels B and E represent pre β-glucuronidase treatment, while panels C and F represent post β-glucuronidase treatment. All analytical conditions are described in the Materials and Methods. Different color tracings represent unique MRM transitions for each metabolite (see Tables 1 and 2 in the Examples).

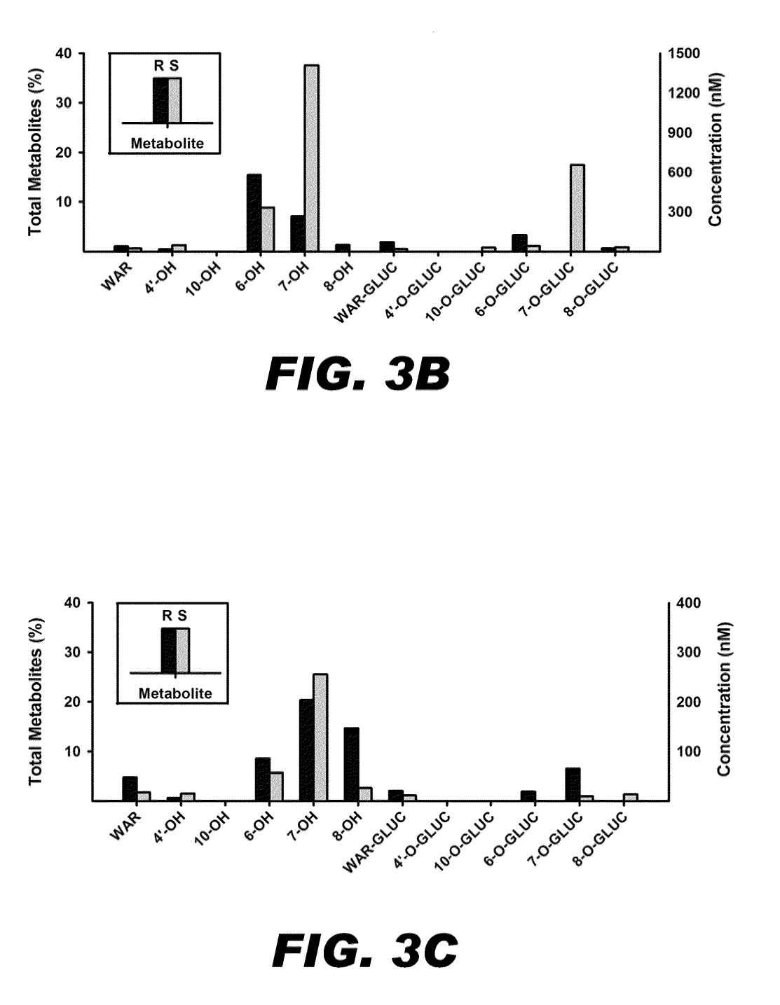

FIG. 3. Metabolic profiles for (A) F84, (B) F85, and (C) F87. Values represent the concentration of each metabolite in urine (nM). Each metabolite is composed of (black) (R)- and (gray) (S)-enantiomers. All analytical conditions and calculations are described in Example 1.

FIG. 4. Sites of hydroxywarfarin glucuronidation catalyzed by human UGTs. Upper) the combined structure of 8-, 7-, 6-, 10-, and 4′-hydroxywarfarin. Lower) putative glucuronide products formed by indicated UGT isoforms. Size of font used for each isoform represents relative levels of activity of each in specific reaction.

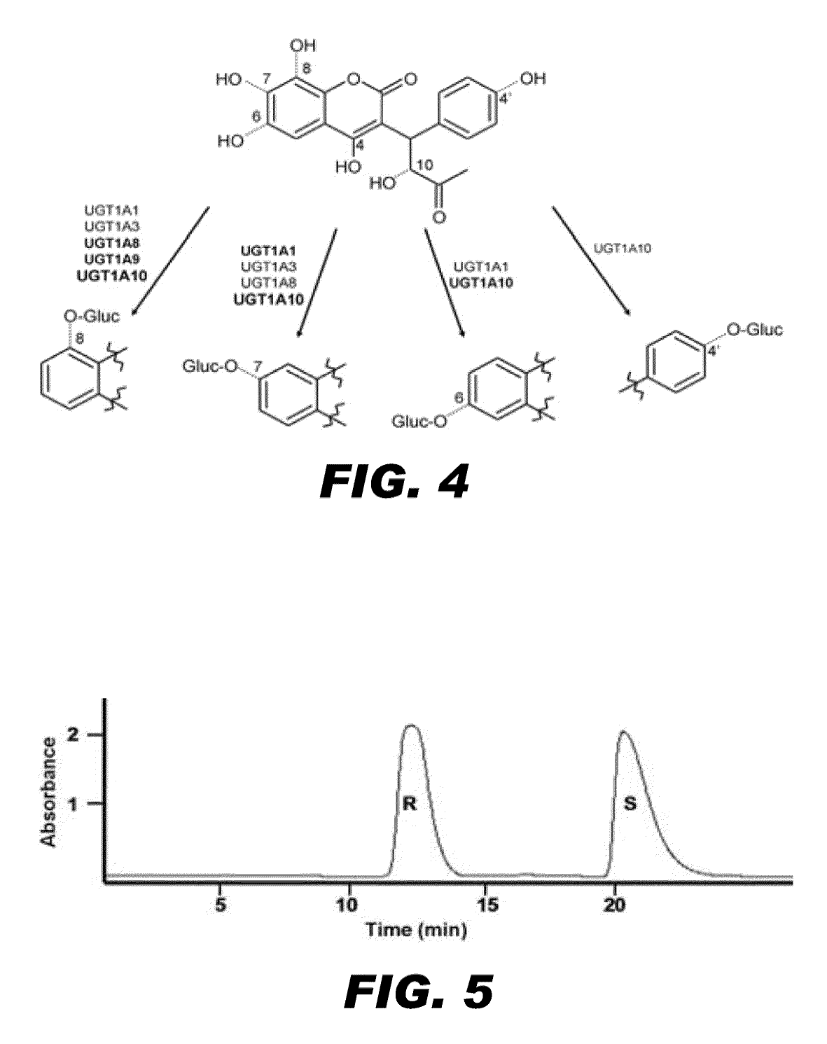

FIG. 5. Separation of R- and S-7-OHWar by HPLC.

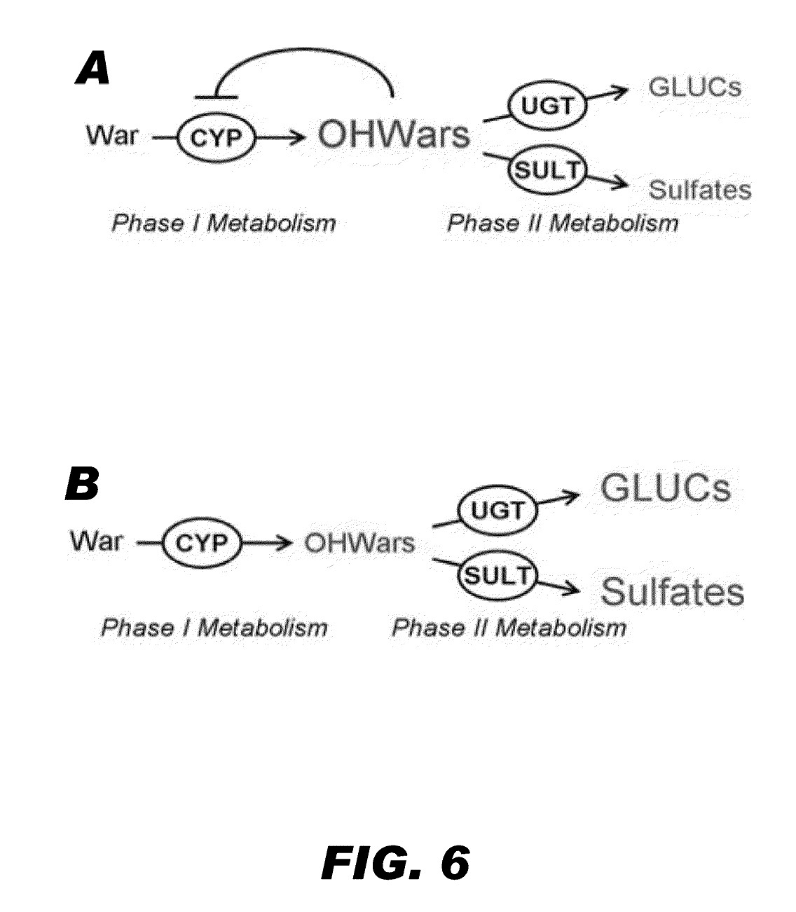

FIG. 6. A) CYP feedback inhibition mechanism B) Suppressed inhibition by Phase II enzymes.

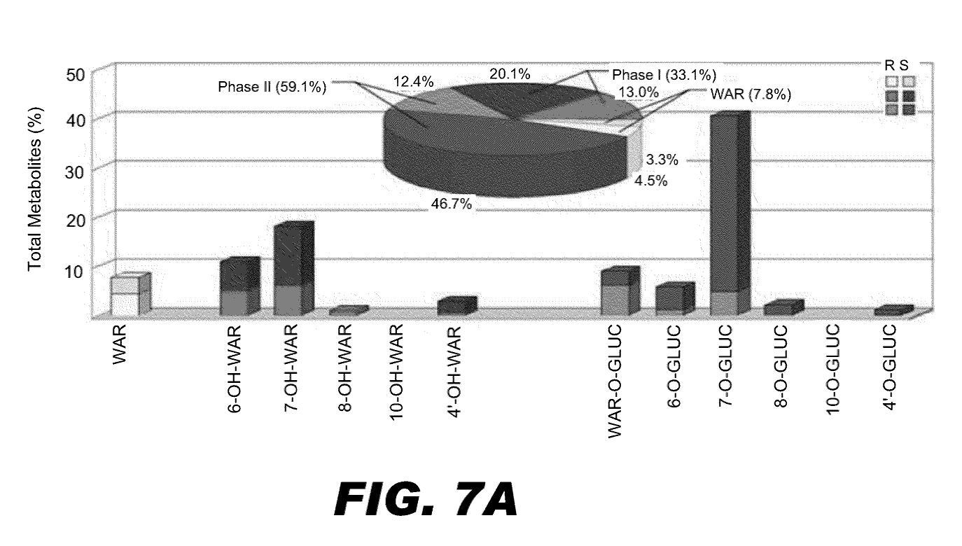

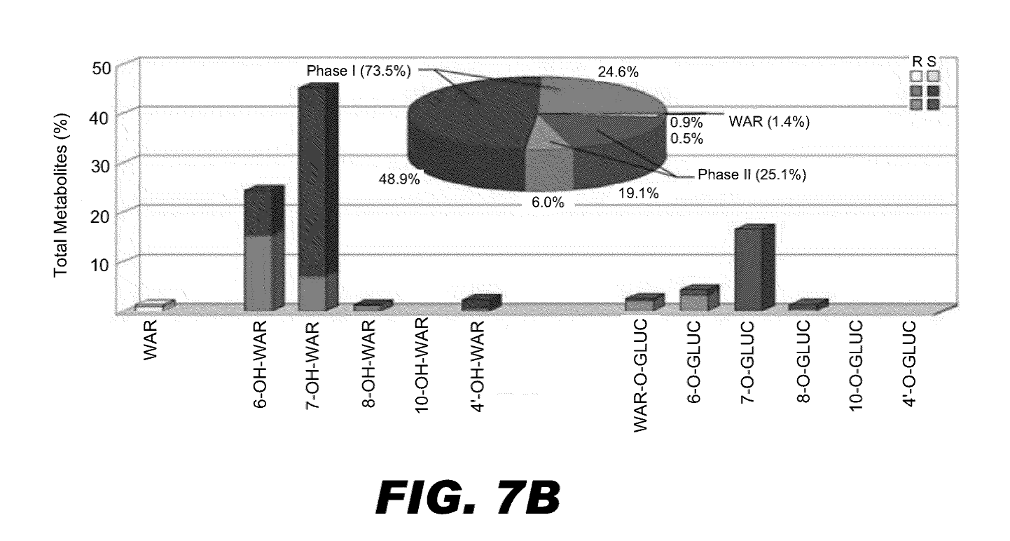

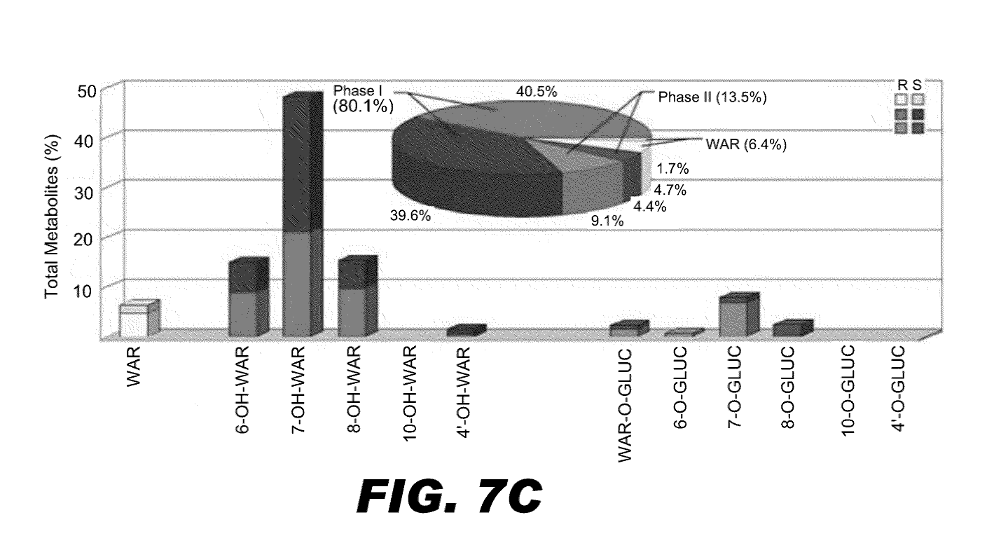

FIG. 7. Urinary metabolic profiles for three patients. A) 84 y.o. Female, B) 85 y.o. Female, and C) 87 y.o. Female.

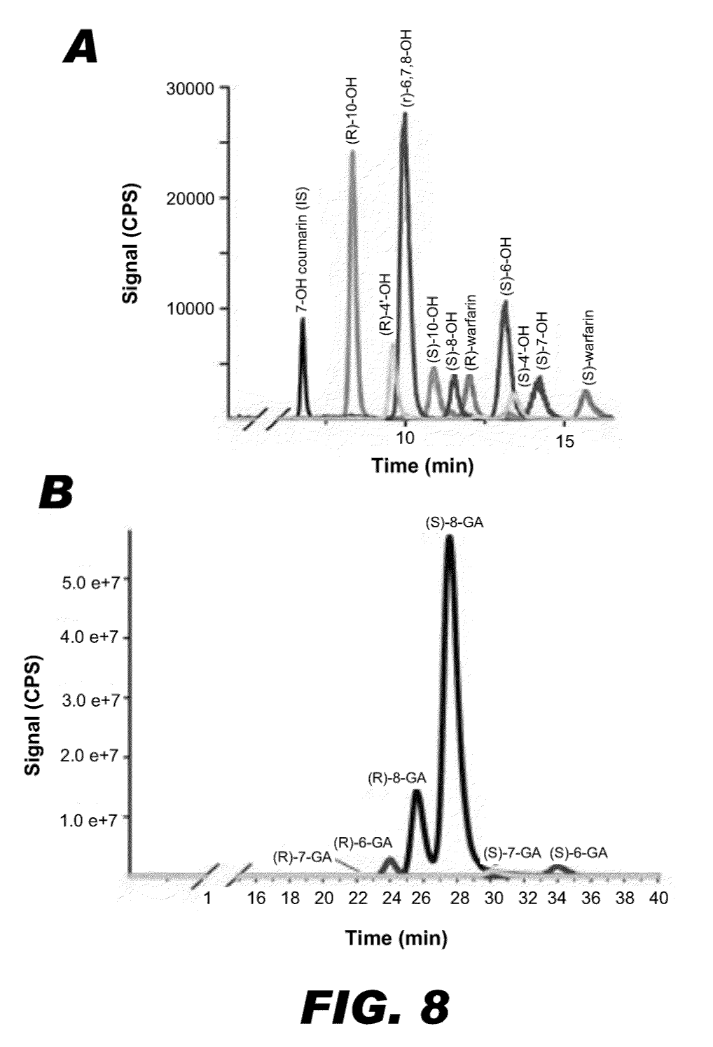

FIG. 8. Sample chromatograms of OHWars (A) and Glucs (B).

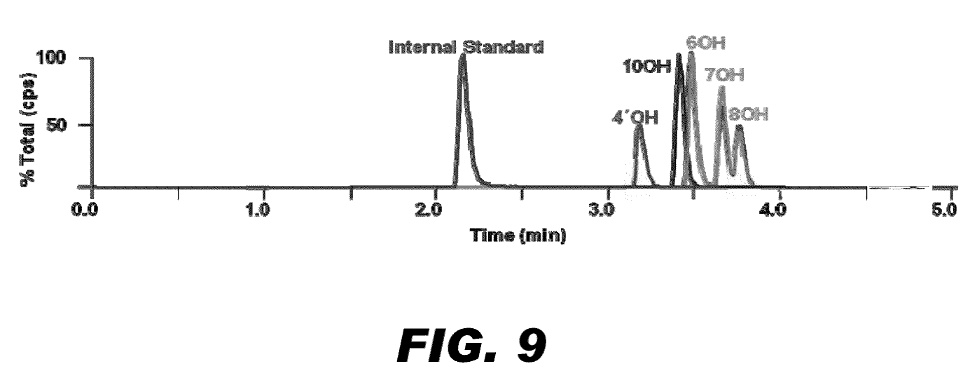

FIG. 9. UPLC-MS/MS chromatogram of hydroxywarfarin standards. 4′-(purple), 10-(blue), 6-(gold), 7-(gold), and 8-hydroxywarfarin (gold) were present at 200 nM in this sample, while the internal standard, 7-hydroxycoumarin (red) was present at 20 μM. Each color tracing represents a unique SRM. Each hydroxywarfarin achieved baseline separation with the exception of 7- and 8-hydroxywarfarin which showed minor co-elution. CPS=counts per second.

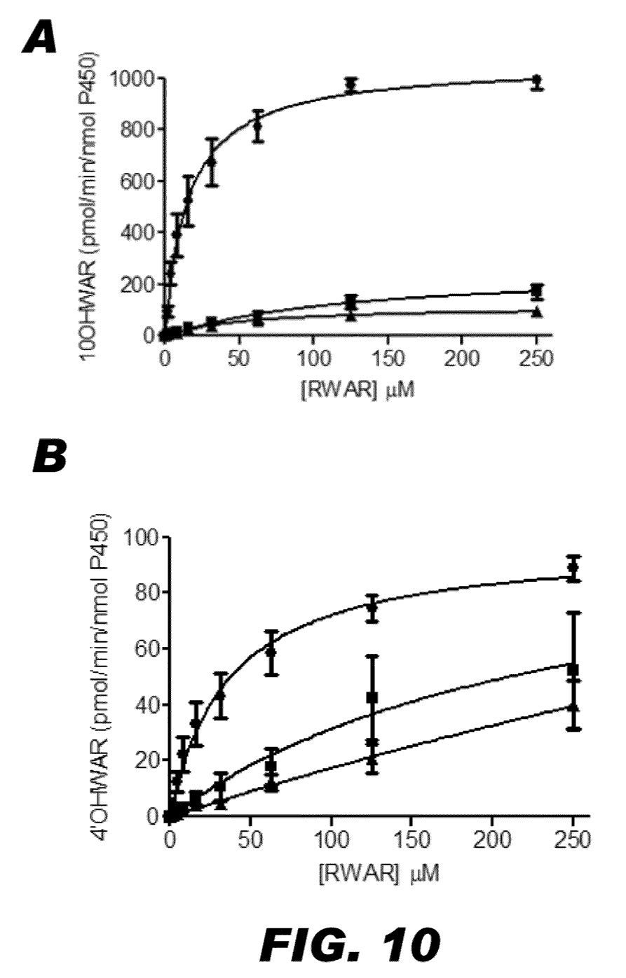

FIG. 10. Steady-state metabolism of R-warfarin by CYP3A4, CYP3A5, and CYP3A7. Circles represent CYP3A4, Squares represent CYP3A5, and triangles represent CYP3A7. A total of nine substrate (S-warfarin) concentrations were used varying from 0 to 250 μM. Data reflect four experimental replicates. The kinetics of 10-hydroxywarfarin (Panel A) and 4′-hydroxywarfarin (Panel B) product formation were fit to the Michaelis-Menten equation with error bars indicating standard error of the mean. Reactions were performed at 370 C in 50 mM potassium phosphate buffer (pH 7.4). See Materials and Methods for further details.

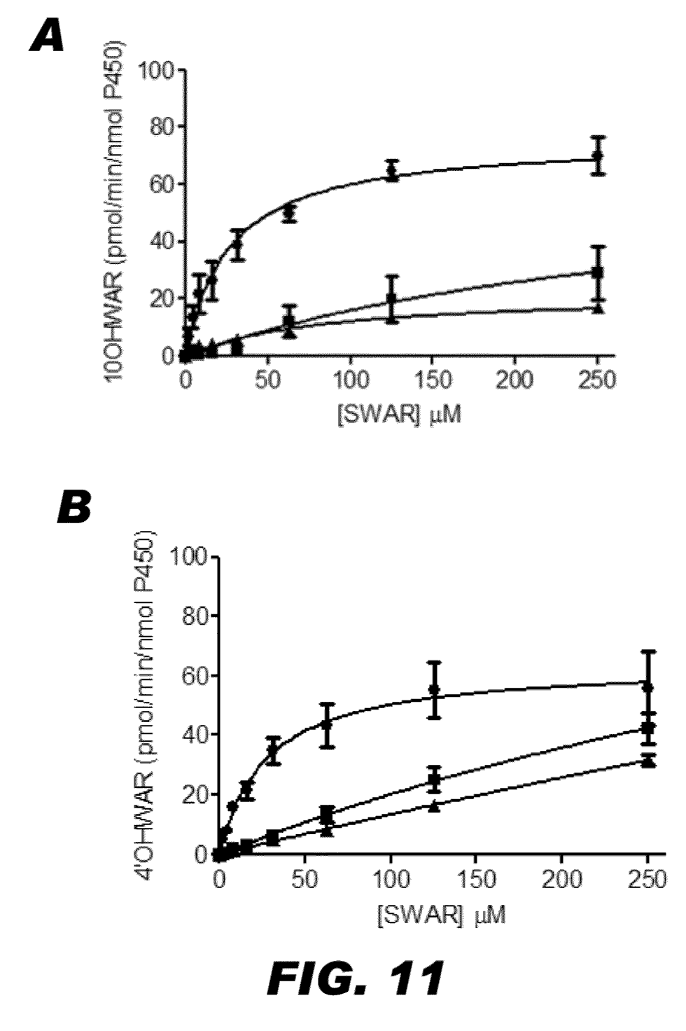

FIG. 11. Steady-state metabolism of 5-warfarin by CYP3A4, CYP3A5, and CYP3A7. Circles represent CYP3A4, Squares represent CYP3A5, and triangles represent CYP3A7. A total of nine substrate (S-warfarin) concentrations were used varying from 0 to 250 μM. Data reflect four experimental replicates. The kinetics of 10-hydroxywarfarin (Panel A) and 4′-hydroxywarfarin (Panel B) product formation were fit to the Michaelis-Menten equation with error bars indicating standard error of the mean. Reactions were performed at 370 C in 50 mM potassium phosphate buffer (pH 7.4).

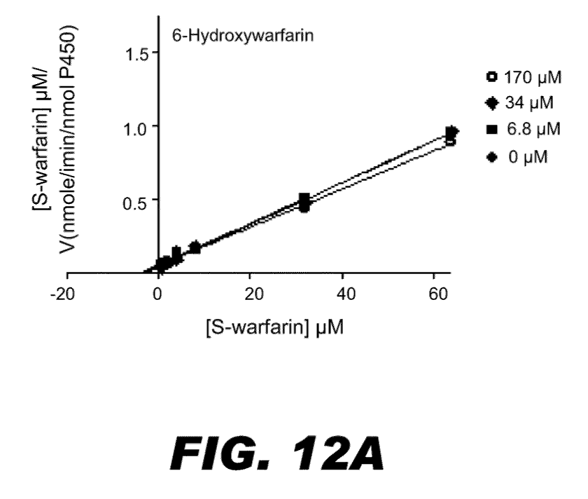

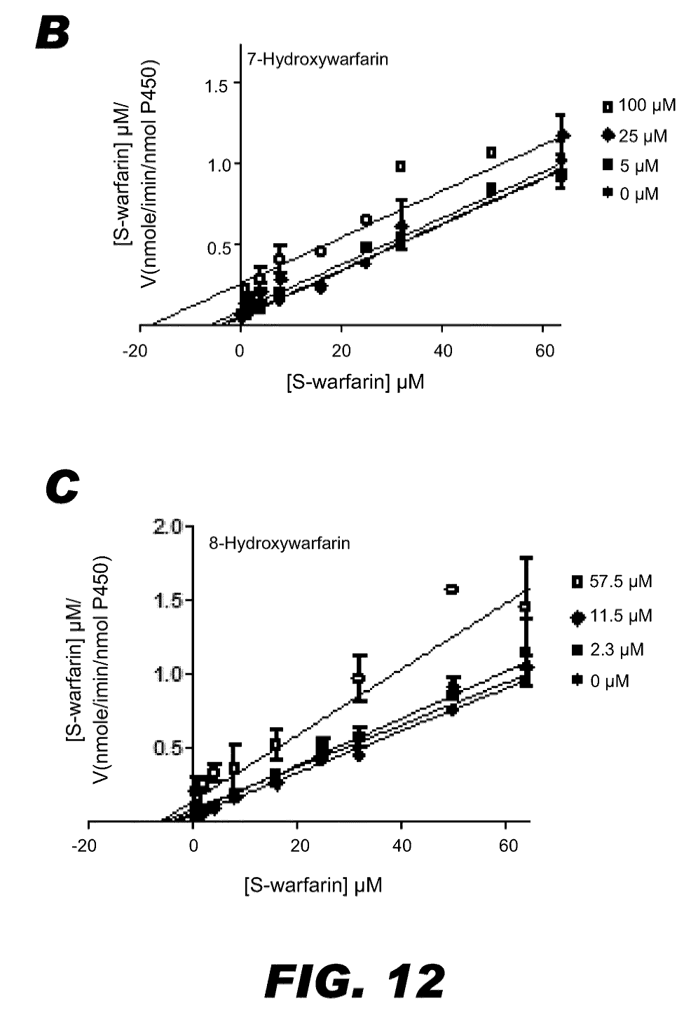

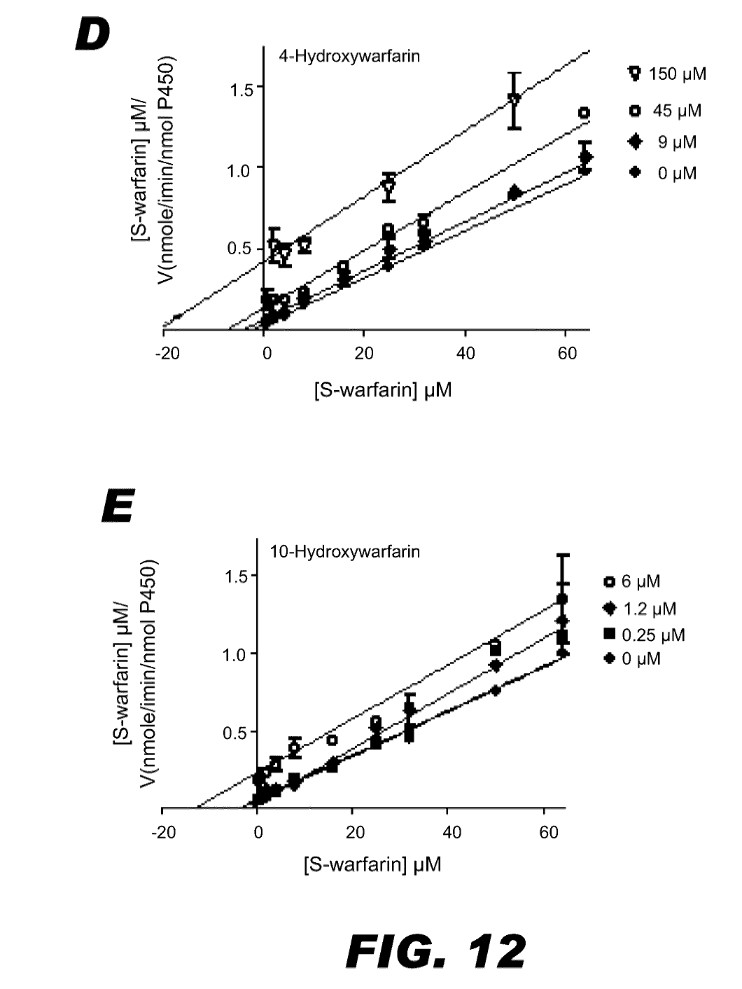

FIG. 12. Hanes-Woolf plots of hydroxywarfarin inhibition of S-warfarin metabolism by recombinant CYP2C9. Linear plots represent the Hanes-Woolf transformation of the DynaFit non-linear regression for the reported model of inhibition for each hydroxywarfarin inhibitor (panels A-E). Individual tracings represent different concentrations of hydroxywarfarin inhibitors as shown in each legend. Reactions were performed at least four times at 3° C. and pH 7.4.

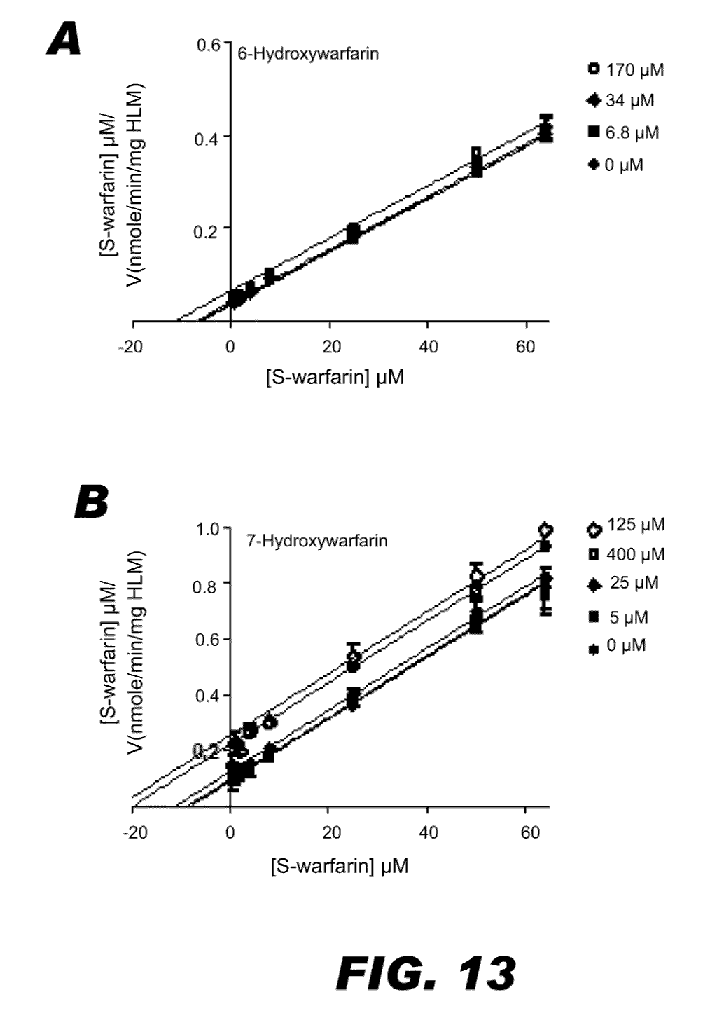

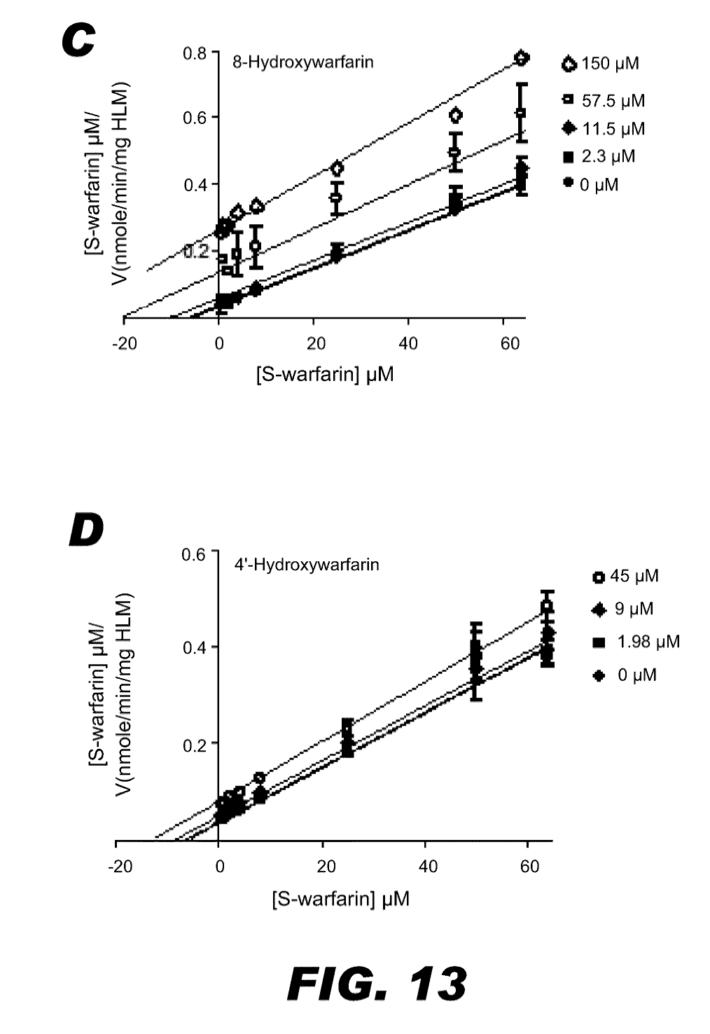

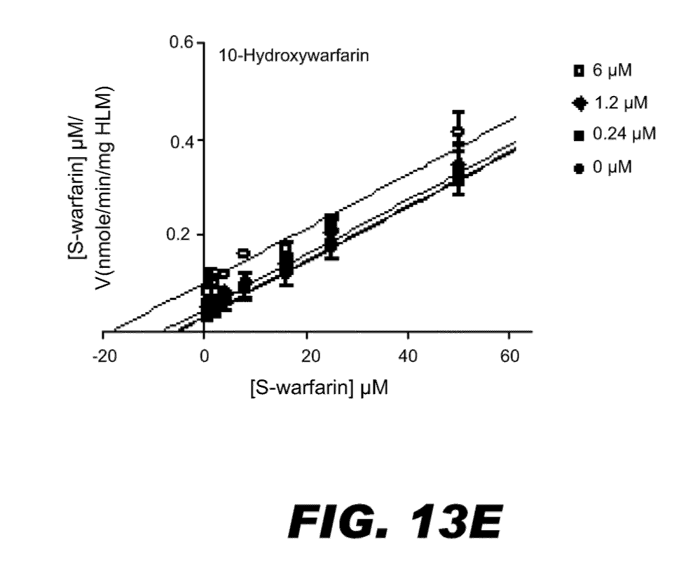

FIG. 13. Hanes-Woolf plots of hydroxywarfarin inhibition of S-warfarin metabolism by human liver microsomes. Linear plots represent the Hanes-Woolf transformation of the DynaFit non-linear regression for the competitive model of inhibition for each hydroxywarfarin inhibitor (panels A-E). Individual tracings represent different concentrations of hydroxywarfarin inhibitors as shown in each legend. Reactions were performed at least four times at 37° C. and pH 7.4.

### DETAILED DESCRIPTION OF THE INVENTION

The present invention encompasses a metabolic profile of a pharmaceutical compound in a sample from a subject. Additionally, the invention further encompasses a method for determining a metabolic profile and uses for such a profile. Advantageously, a subject's metabolic profile may be used to determine the effective dose of a pharmaceutically active compound for the subject.

### I. Metabolic Profile

One aspect of the present invention encompasses a metabolic profile of a pharmaceutical compound in a sample from a subject. As used herein, a “metabolic profile” refers to the identity and stereochemistry of at least one metabolite of a pharmaceutical compound in a sample from a subject. In an exemplary embodiment, the metabolic profile is stored on a computer-readable medium. A metabolic profile may comprise phase I metabolites and/or phase II metabolites. Phase I metabolites are the products of phase I metabolic reactions. Phase I metabolic reactions generally introduce or unmask polar groups, which results in (more) polar metabolites of the original compound. Phase II reactions, also known as conjugation reactions (e.g., with glucuronic acid, sulfonates, glutathione or amino acids) generally involve interactions with the polar functional groups of phase I metabolites.

In one embodiment, the invention provides a metabolic profile of warfarin in a subject. A metabolic profile of warfarin may comprise products of phase I and/or phase II warfarin metabolism. In certain embodiments, a metabolic profile of warfarin may comprise one or more compounds selected from the group comprising warfarin, hydroxywarfarins, and conjugates of hydroxywarfarins. Non-limiting examples of hydroxywarfarins may include 6-hydroxywarfarin, 7-hydroxywarfarin, 8-hydroxywarfarin, 10-hydroxywarfarin, and 4′-hydroxywarfarin. Non-limiting examples of conjugates of hydroxywarfarin may include glucuronides and sulfates. In some embodiments, the conjugates comprise a single glucuronide, whereas in other embodiments, the conjugates may comprise one or more sulfate groups. In other embodiments, the conjugates may comprise a combination of bis- or di-sulfated metabolite of warfarin, any hydroxywarfarin derivative, or any warfarin alcohol derivative, or any combination of bis- or di-glucuronidated metabolite of warfarin, any hydroxywarfarin derivative, or any warfarin alcohol derivative. In each of the above embodiments, the metabolite may have (R) or (S) stereochemistry. In a particular embodiment, a metabolic profile of warfarin may comprise one or more of the compounds listed in Table A.

TABLE A

| | |
|---|---|
| (R) warfarin | (S) warfarin |
| (R) 4′-hydroxywarfarin | (S) 4′-hydroxywarfarin |
| (R) 6-hydroxywarfarin | (S) 6-hydroxywarfarin |
| (R) 7-hydroxywarfarin | (S) 7-hydroxywarfarin |
| (R) 8-hydroxywarfarin | (S) 8-hydroxywarfarin |
| (R) 10-hydroxywarfarin | (S) 10-hydroxywarfarin |
| (R) warfarin-GLUC | (S) warfarin-GLUC |
| (R) 4′-hydroxywarfarin-GLUC | (S) 4′-hydroxywarfarin-GLUC |
| (R) 6-hydroxywarfarin-GLUC | (S) 6-hydroxywarfarin-GLUC |
| (R) 7-hydroxywarfarin-GLUC | (S) 7-hydroxywarfarin-GLUC |
| (R) 8-hydroxywarfarin-GLUC | (S) 8-hydroxywarfarin-GLUC |
| (R) 10-hydroxywarfarin-GLUC | (S) 10-hydroxywarfarin-GLUC |
| (R) warfarin-sulfate | (S) warfarin-sulfate |
| (R) 4′-hydroxywarfarin-sulfate | (S) 4′-hydroxywarfarin-sulfate |
| (R) 6-hydroxywarfarin-sulfate | (S) 6-hydroxywarfarin-sulfate |
| (R) 7-hydroxywarfarin-sulfate | (S) 7-hydroxywarfarin-sulfate |
| (R) 8-hydroxywarfarin-sulfate | (S) 8-hydroxywarfarin-sulfate |
| (R) 10-hydroxywarfarin-sulfate | (S) 10-hydroxywarfarin-sulfate |

In an exemplary embodiment, a metabolic profile of the invention further comprises the quantity of each metabolite identified. A method for determining the identity, stereochemistry, and quantity of warfarin metabolites is detailed in section I(b) below, and in the Examples.

Suitable samples for determining a metabolic profile may include tissue samples and fluid samples. For instance, non-limiting examples of fluid samples may include urine samples, plasma samples, whole-blood samples, and serum samples. Non-limiting examples of tissue samples may include biopsy samples, fresh frozen samples, or other tissue samples from a subject. In certain embodiments, the tissue samples are liver samples or intestinal samples. In an exemplary embodiment, the sample is a urine or plasma sample.

(a) database

The invention also encompasses a database. Generally speaking, a database of the invention comprises at least one metabolic profile of a subject, and is typically stored on a computer-readable medium. A database may comprise more than one metabolic profile for a given subject over time (a “subject” database). Alternatively, a database may comprise a metabolic profile of a pharmaceutically active compound in a subject correlated to an effective dose range for the compound in the subject (a “dose” database). For instance, the database may comprise at least one warfarin metabolic profile of a subject, wherein the profile is correlated to one or more effective warfarin doses for the subject. Each of these databases is discussed in more detail below.

A database of the invention may also comprise background data on a subject. Non-limiting examples of background data may include age, weight, gender, race, ethnicity, diet, socioeconomic status, current and/or past medications the subject has been exposed to, and the health status of the subject, including diseases or disorders that the subject may have.

i. Subject Database

A subject database may comprise at least one metabolic profile of one or more pharmaceutically active compounds in a subject over time. For example, a subject database may comprise a metabolic profile from 2, 3, 4, 5, 6, 7, 8, 9, 10, 11, 12, 13, 14, 15, 16, 17, 18, 19, 20, or more than 20 time points for each pharmaceutically active compound. In one embodiment, the invention encompasses a subject database that comprises a warfarin metabolic profile for the subject from at least two different time points. In another embodiment, the invention encompasses a subject database that comprises a warfarin metabolic profile for the subject from at least 3, 4, 5, 6, 7, 8, 9, 10, 11, 12, 13, 14, 15, 16, 17, 18, 19, 20, or more than 20 time points. The time points may be minutes apart, hours apart, days apart, months apart, or years apart.

In a further embodiment, a database of the invention may comprise multiple subject databases.

ii. Dose Database

As stated above, a dose database may comprise a metabolic profile of a pharmaceutically active compound in a subject correlated to an effective dose range for the compound in the subject. Such a database may be used for determining the effective dose of a pharmaceutically active compound as detailed in section I(c) below. A dose database may comprise a metabolic profile of a pharmaceutically active compound correlated to an effective dose range for the compound for at least 1, 2, 3, 4, 5, 6, 7, 8, 9, 10, or more than 10 subjects. In certain embodiments, a database may comprise at least 20, 30, 40, 50, 60, 70, 80, 90, 100, or more than 100 subjects. In other embodiments, a database may comprise at least 100, 200, 300, 400, 500, 600, 700, 800, 900, 1000, or more than 1000 subjects.

One embodiment of the invention encompasses a warfarin dose database. A warfarin dose database comprises at least one warfarin metabolic profile of a subject, wherein the profile is correlated to one or more effective warfarin doses for the subject. In one embodiment, a warfarin dose database is comprised of phase I warfarin metabolic profiles. In yet another embodiment, a warfarin dose database is comprised of phase II warfarin metabolic profiles. In still another embodiment, a warfarin dose database is comprised of phase I and phase II warfarin metabolic profiles.

### (b) Methods of Determining a Metabolic Profile

Another aspect of the present invention encompasses a method for determining a metabolic profile of a subject. The method comprises determining the identity and stereochemistry of at least one metabolite of a pharmaceutically active compound in a sample from a subject. For instance, in some embodiments, the method may be used to determine the identity and stereochemistry of at least one phase I metabolite of a pharmaceutically active compound. In other embodiments, the method may be used to determine the identity and stereochemistry of at least one phase II metabolite of a pharmaceutically active compound. In another embodiment, the method may be used to determine the identity and stereochemistry of at least one phase I and at least one phase II metabolite of a pharmaceutically active compound.

Generally speaking, the identity and stereochemistry of a metabolite may be determined utilizing liquid chromatography followed by mass spectrometry. In some embodiments, two stages of liquid chromatography followed by mass spectrometry may be used. The first stage may be used to separate the metabolites while the second stage may be used to separate the stereoisomers of the metabolites. In other embodiments, only a single stage is used.

In some embodiments, the liquid chromatography is high performance liquid chromatography (HPLC). Non-limiting examples of HPLC may include partition chromatography, normal phase chromatography, displacement chromatography, reverse phase chromatography, size exclusion chromatography, ion exchange chromatography, bioaffinity chromatography, or aqueous normal phase chromatography. In one embodiment, the liquid chromatography may be reverse phase HPLC. The mobile phase used in the HPLC may be a gradient or may be isocratic.

In certain embodiments, the mass spectrometry may be tandem mass spectrometry. In some embodiments, the mass spectrometry may be quadrupole mass spectrometry. In alternative embodiments, other detection means may be employed, such as fluorescence, UV-Vis, or a chiral detector. In an exemplary embodiment, the method comprises HPLC followed by tandem mass spectrometry. In a further exemplary embodiment, the method may comprise HPLC followed by quadrupole tandem mass spectrometry. In each of the above embodiments, the liquid chromatography followed by mass spectrometry may also be used to determine the quantity of an identified metabolite in a metabolic profile.

Suitable samples for the method may include tissue samples and fluid samples from a subject. For instance, non-limiting examples of fluid samples may include urine samples, plasma samples, whole-blood samples, and serum samples. Non-limiting examples of tissue samples may include biopsy samples, fresh frozen samples, or other tissue samples from a subject. For instance, liver and/or intestinal samples may be suitable. In an exemplary embodiment, the sample is a urine or plasma sample. Generally speaking, the pharmaceutically active compound should be administered to the subject before the sample is collected. In some embodiments, the compound should be administered to the subject minutes, hours, days, weeks, and/or months before a sample is collected.

In particular embodiments, a method of the invention may be used to determine a warfarin metabolic profile in a sample from a subject. In one embodiment, the method comprises determining the identity and stereochemistry of warfarin and/or one or more phase I metabolites of warfarin (e.g. hydroxywarfarins) in a sample from a subject. In another embodiment, the method comprises determining the identity and stereochemistry of one or more phase II metabolites of hydroxywarfarins, such as glucuronides and sulfates. In yet another embodiment, the method comprises determining the identity and stereochemistry of warfarin, one or more phase I metabolites, and one or more phase II metabolites of hydroxywarfarins. In a further embodiment, the method comprises determining the identity and stereochemistry of a compound from Table A in a sample from a subject.

As described above, liquid chromatography followed by mass spectrometry may be used to determine the identity and stereochemistry of a warfarin metabolite. Additionally, liquid chromatography followed by mass spectrometry may be used to determine the quantity of a warfarin metabolite. In some embodiments, two stages of liquid chromatography followed by mass spectrometry may be used. The liquid chromatography portions of the first and second stage are described below. The mass spectrometry settings for the first and second stage are detailed in the Examples.

i. First Stage

The first stage of liquid chromatography followed by mass spectrometry may be used to separate and identify a warfarin metabolite. Typically, the first stage comprises reverse phase HPLC followed by tandem mass spectrometry. The liquid chromatography step of the first stage is typically reverse phase HPLC. The mobile phase for the HPLC is generally a gradient comprised of two different solutions: A and B. In one embodiment, A is comprised of water. A may be slightly acidic or slightly basic. For instance, in an embodiment, A is comprised of water and a weak acid. The percentage of weak acid in A may be from about 1% to about 0.01% v/v. In one embodiment, the percentage of weak acid in A may be about 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, or 0.01% v/v. In another embodiment, the percentage of weak acid in A may be about 0.2% to about 0.08% v/v. In yet another embodiment, the percentage of weak acid in A may be about 0.1% v/v. In an exemplary embodiment, the weak acid may be a weak organic acid, such as acetic acid. In a further exemplary embodiment, A may be 0.1% v/v acetic acid in water.

In one embodiment, B may be an organic solvent miscible in water. Examples of such solvents may include acetonitrile, methanol, THF, or isopropanol. In certain embodiments, B may be methanol. B may be slightly acidic or slightly basic. For instance, in an embodiment, B may be comprised of methanol and a weak acid. The percentage of weak acid in B may be from about 1% to about 0.01% v/v. In one embodiment, the percentage of weak acid in B may be about 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, or 0.01% v/v. In another embodiment, the percentage of weak acid in B may be about 0.2% to about 0.08% v/v. In yet another embodiment, the percentage of weak acid in B may be about 0.1% v/v. In an exemplary embodiment, the weak acid may be a weak organic acid, such as acetic acid. In a further exemplary embodiment, B may be 0.1% v/v acetic acid in methanol.

The gradient of the mobile phase generally varies from about 50% B to about 100% B. In some embodiments, the gradient sequentially comprises (a) about 50% A and about 50% B, (b) a gradient from about 50% to about 90% B, (c) a gradient from about 90% to about 100% B, (d) about 100% B, (e) a gradient from about 100% to about 50% B, and (f) about 50% B. Generally speaking, the flow time for step (a) above is about 25 to about 35 seconds, the flow time for step (b) is about 25 seconds to about 6 min, the flow time for step (c) is about 5 min twenty-five seconds to about 5 minutes forty seconds, the flow time for step (d) is about two minutes to about three minutes, the flow time for step (e) is about seven min thirty seconds to about eight minutes thirty seconds, and the flow time for step (f) is about one minute thirty seconds to about two minutes thirty seconds.

In certain embodiments, the gradient comprises about 50% A and about 50% B for about the first 30 s, a linear gradient from about 50% to about 90% B (for about 0.5 to about 5.5 min), a linear gradient from about 90% to about 100% B (for about 5.5 to about 5.6 min), about 100% B maintained for about 2.5 min, about 100% to about 50% B (for about 7.9 to about 8.0 min), and about 50% B maintained for about 2 min.

Usually, the flow time for the first stage is less than 20 min. In some embodiments, the flow time for the first stage is about 20, 19, 18, 17, 16, 15, 14, 13, 12, 11, 10, or less than 10 minutes. In some embodiments, the flow rate may be between about 700 uL/min and about 1200 uL/min. In other embodiments, the flow rate for the first stage may be about 1 mL/min.

The temperature for the first and second stage may typically be between about 25° C. and 50° C. In one embodiment, the temperature may be about 35, 36, 37, 38, 39, 40, 41, 42, 43, 44, or 45° C. In another embodiment, the temperature may be about 40° C.

ii. Second Stage

The second stage of liquid chromatography followed by mass spectrometry may determine the stereochemistry of the metabolites identified in the first stage. In particular, the second stage may determine whether the metabolite has (R) or (S) stereochemistry. Usually, the liquid chromatography of the second stage is also HPLC. The mobile phase is typically isocratic, and may be comprised of two solutions: A and B. In one embodiment, A is comprised of water. A may be slightly acidic or slightly basic. For instance, in an embodiment, A is comprised of water and a weak acid. The percentage of weak acid in A may be from about 1% to about 0.01% v/v. In one embodiment, the percentage of weak acid in A may be about 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, or 0.01% v/v. In another embodiment, the percentage of weak acid in A may be about 0.2% to about 0.08% v/v. In yet another embodiment, the percentage of weak acid in A may be about 0.1% v/v. In an exemplary embodiment, the weak acid may be a weak organic acid, such as acetic acid. In a further exemplary embodiment, A may be 0.1% v/v acetic acid in water.

In one embodiment, B may be an organic solvent miscible in water. Examples of such solvents may include acetonitrile, methanol, THF, or isopropanol. In certain embodiments, B may be acetonitrile. B may be slightly acidic or slightly basic. For instance, in an embodiment, B may be comprised of acetonitrile and a weak acid. The percentage of weak acid in B may be from about 1% to about 0.01% v/v. In one embodiment, the percentage of weak acid in B may be about 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, or 0.01% v/v. In another embodiment, the percentage of weak acid in B may be about 0.2% to about 0.08% v/v. In yet another embodiment, the percentage of weak acid in B may be about 0.1% v/v. In an exemplary embodiment, the weak acid may be a weak organic acid, such as acetic acid. In a further exemplary embodiment, B may be 0.1% v/v acetic acid in acetonitrile.

The isocratic mobile phase may comprise a ratio of A:B between about 75:25 and about 85:15. In some embodiments, the ratio may be about 75:25, 76:24, 77:23, 78:22, 79:21, 80:20, 81:19, 82:18, 83:17, 84:16, or 85:15. In certain embodiments, the ratio may be about 80:20.

Usually, the flow time for the second stage is less than 20 min. In some embodiments, the flow time for the second stage is about 20, 19, 18, 17, 16, 15, 14, 13, 12, 11, 10, or less than 10 minutes. Alternatively, the total flow time for the first and second stages combined is generally less than 25 min. For instance, the total flow time may be less than 24, 23, 22, 21, 20, 19, 18, 17, 16, or less than 15 min. In some embodiments, the flow rate for the second stage may be between about 600 uL/min and about 100 uL/min. In other embodiments, the flow rate for the second stage may be about 800 uL/min.

### (c) Uses for a Metabolic Profile

Still another aspect of the invention encompasses uses for a metabolic profile. Non-limiting examples of uses for a metabolic profile are detailed below.

i. Determining an Effective Dose Range of a Pharmaceutically Active Compound

In one embodiment, the invention provides a method for determining an effective dose range of a pharmaceutically active compound. The method comprises, in part, determining the metabolic profile of a subject for a pharmaceutically active compound. Then the profile may be compared to a dose database for the particular pharmaceutically active compound. Stated another way, the profile may be compared to a database comprising at least one metabolic profile for the pharmaceutically active compound correlated to a known effective dose of the compound. A database profile similar to the subject's profile may then be selected, wherein the effective dose range correlated to the database profile is the effective dose range for the subject.

In one embodiment, the invention provides a method for determining an effective dose range of warfarin for a subject. The method comprises, in part, determining the warfarin metabolic profile of a subject. Then the profile may be compared to a warfarin dose database. Stated another way, the profile may be compared to a database comprising at least one warfarin metabolic profile correlated to a known effective dose of warfarin. A database profile similar to the subject's profile may then be selected, wherein the effective dose range correlated to the database profile is the effective dose range for the subject.

The warfarin metabolic profile of a subject, as detailed above, may be determined by administrating warfarin to the subject, collecting a sample from the subject, and determining the identity and stereochemistry of at least one warfarin metabolite. In one embodiment, the sample is collected from the subject minutes after administration of warfarin. In another embodiment, the sample is collected hours after administration of warfarin. In an exemplary embodiment, the sample is collected before a difference in coagulation due to the warfarin administration is detectable.

In certain embodiments, the warfarin metabolic profile comprises at least one phase I warfarin metabolite. In other embodiments, the warfarin metabolic profile comprises at least one phase II warfarin metabolite. In one embodiment, the warfarin metabolic profile comprises warfarin, at least one phase I warfarin metabolite, and at least one phase II warfarin metabolite.

ii. Screening for Compounds that Modulate the Metabolism of a Pharmaceutically Active Compound

Another embodiment of the invention comprises a method of screening for compounds that modulate the metabolism of a pharmaceutically active compound. The method typically comprises determining the metabolic profile of a first pharmaceutically active compound in a subject, administering a second compound to the subject, and detecting a change in the metabolic profile of the first pharmaceutically active compound. A change in the profile indicates that the second compound modulates the metabolism of the first compound. In certain embodiments, the second compound may be another pharmaceutically active compound, a food ingredient, an environmental compound, or a metabolite of the first compound.

In one embodiment, the invention provides a method of screening for compounds that modulate warfarin metabolism in a subject. The method comprises determining the warfarin metabolic profile of a subject, administering a compound to the subject, and detecting a change in the warfarin metabolic profile. A change in the profile indicates that the compound modulates the metabolism of warfarin. As detailed above, the compound may be a pharmaceutically active compound, a food ingredient, an environmental compound, or a warfarin metabolite.

The change in the profile may be a change in the presence and/or absence of a metabolite, or may be a change in the quantity and/or ratio among the metabolites.

iii. Detecting Changes in a Pharmaceutically Active Compound's Metabolism

Yet another embodiment of the invention encompasses a method for determining changes in the metabolism of a pharmaceutically active compound in a subject over time. The method generally comprises determining a metabolic profile for the compound in a subject at a first time point and at least one other time point. The profiles from the two time points may be compared, such that a change in the profile may indicate a change in the metabolism of the pharmaceutically active compound. In one embodiment, the pharmaceutically active compound is warfarin. In some embodiments, the method may comprise comparing profiles from 2, 3, 4, 5, 6, 7, 8, 9, 10, or more time points. The time points may be days apart, weeks apart, months apart, or years apart.

The profiles from the first and any subsequent time points may be stored in a subject database, as described above.

### II. Warfarin Metabolites

A further aspect of the invention encompasses warfarin metabolites. In particular, the invention encompasses phase II warfarin metabolites. For example, the invention encompasses hydroxywarfarin glucuronide conjugates and hydroxywarfarin sulfate conjugates. In other embodiments, the conjugates may comprise a combination of bis- or di-sulfated metabolite of warfarin, any hydroxywarfarin derivative, or any warfarin alcohol derivative, or any combination of bis- or di-glucuronidated metabolite of warfarin, any hydroxywarfarin derivative, or any warfarin alcohol derivative. In one embodiment, the invention encompasses the phase II warfarin metabolites listed in Table B.

TABLE B

| | |
|---|---|
| (R) warfarin-GLUC | (S) warfarin-GLUC |
| (R) 4′-hydroxywarfarin-GLUC | (S) 4′-hydroxywarfarin-GLUC |
| (R) 6-hydroxywarfarin-GLUC | (S) 6-hydroxywarfarin-GLUC |
| (R) 7-hydroxywarfarin-GLUC | (S) 7-hydroxywarfarin-GLUC |
| (R) 8-hydroxywarfarin-GLUC | (S) 8-hydroxywarfarin-GLUC |
| (R) 10-hydroxywarfarin-GLUC | (S) 10-hydroxywarfarin-GLUC |
| (R) warfarin-sulfate | (S) warfarin-sulfate |
| (R) 4′-hydroxywarfarin-sulfate | (S) 4′-hydroxywarfarin-sulfate |
| (R) 6-hydroxywarfarin-sulfate | (S) 6-hydroxywarfarin-sulfate |
| (R) 7-hydroxywarfarin-sulfate | (S) 7-hydroxywarfarin-sulfate |
| (R) 8-hydroxywarfarin-sulfate | (S) 8-hydroxywarfarin-sulfate |
| (R) 10-hydroxywarfarin-sulfate | (S) 10-hydroxywarfarin-sulfate |

The invention also comprises uses of warfarin metabolites. In one embodiment, the invention provides a method for decreasing blood clot formation in a subject. The method generally comprises administering a warfarin metabolite to the subject. In some embodiments, the warfarin metabolite is a hydroxywarfarin. In other embodiments, the warfarin metabolite is a phase II metabolite. In still other embodiments, the warfarin metabolite is a pure warfarin enantiomer.

In another embodiment, the invention encompasses a method for inhibiting a P450 enzyme. The method typically comprises administering a warfarin metabolite to the subject. In some embodiments, the warfarin metabolite is a hydroxywarfarin.

In yet another embodiment, the invention encompasses labeled standards for use in mass spectrometry. Such standards may comprise a warfarin metabolite labeled with a mass spectrometry compatible label, such as deuterium or C13. In particular embodiments, the standards maybe be phase II metabolites of warfarin.

### Definitions

As used herein, “computer-readable medium” refers to a medium that participates in providing instructions to a processor for execution. Such a medium may take many forms, including but not limited to non-volatile media, volatile media, and transmission media. Non-volatile media may include, for example, optical or magnetic disks. Volatile media may include dynamic memory. Transmission media may include coaxial cables, copper wire and fiber optics. Transmission media may also take the form of acoustic, optical, or electromagnetic waves, such as those generated during radio frequency (RF) and infrared (IR) data communications. Common forms of computer-readable media include, for example, a floppy disk, a flexible disk, hard disk, magnetic tape, or other magnetic medium, a CD-ROM, CDRW, DVD, or other optical medium, punch cards, paper tape, optical mark sheets, or other physical medium with patterns of holes or other optically recognizable indicia, a RAM, a PROM, and EPROM, a FLASH-EPROM, or other memory chip or cartridge, a carrier wave, or other medium from which a computer can read.

As used herein, “effective dose” refers to the dose range of a pharmaceutically active compound that results in a desired pharmaceutical effect, without causing undue harm to the subject.

As used herein, “metabolite” refers to a product of the metabolism of the compound. In certain embodiments where the parent compound may be found in a sample with metabolic products of the compound, the parent compound may also be referred to as a metabolite.

As used herein, the labels “(R)” and “(S)” refer to rectus and sinister, respectively, in reference to specific stereoisomers of a compound. As is known in the art, when the chiral center of a molecule is oriented so that the lowest-priority of the four substituents is pointed away from a viewer, if the priority of the remaining three substituents decreases in clockwise direction, it is labeled R (for Rectus). Alternatively, if it decreases in counterclockwise direction, it is labeled S (for Sinister). Substituents are each assigned a priority, according to the Cahn Ingold Prelog priority rules (CIP), based on atomic number.

As used herein, “subject” refers to a mammal. In some embodiments, a suitable subject may include a laboratory animal, a companion animal, a livestock animal, a non-human primate, or a human. In an exemplary embodiment, a subject is a mammal that metabolizes a pharmaceutically active compound of interest. For example, to determine a warfarin metabolic profile in a subject, the subject typically should metabolize warfarin. The subject may be an infant, an adolescent, or an adult.

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that may changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### Examples

The following examples illustrate various iterations of the invention.

### Example 1

Despite potential complications during treatment, warfarin (Coumadin) remains one of the most widely prescribed oral anticoagulants in the Western world. Patients receive warfarin as a racemic mixture consisting of both (R)-and (S)-forms, which differ in potency and metabolic processing. Initial dosages are typically determined by considering age and underlying conditions and, more recently, by identifying genetic factors (1). Approximately 92% of the administered dose is thought to be excreted through urinary pathways (2), and (S)-warfarin is about 3-5 times more potent than (R)-warfarin (3). Medical management of this drug is further complicated by warfarin having a very narrow therapeutic window and wide interindividual variability. Underdosing leads to thrombosis, while overdosing causes dangerous bleeding episodes.

Warfarin metabolism alters its structure and consequently impacts anticoagulant activity and bioavailability; hence, an understanding of these processes is critical for developing better anticoagulant treatment strategies. Cytochrome P450s (P450 or CYP for a particular isoform) introduce a hydroxyl group at one of five positions on warfarin to yield 6-, 7-, 8-, 10-, and 4′-hydroxywarfarins (OH-WAR) (FIG. 1). The formation of each of these compounds is dependent on the particular P450, and typically, each P450 generates multiple products at defined ratios. (S)-Warfarin is primarily hydroxylated by CYP2C9 with minor contributions from CYP2C8, CYP2C18, and CYP2C19 (4-6). By contrast, (R)-warfarin undergoes hydroxylation by CYP1A2 and CYP3A4 (4, 5, 7) and may serve as a substrate for CYP1A1, CYP2C8, CYP2C18, CYP2C19, and CYP3A4 (4, 5, 7).

Unlike P450 reactions, phase II metabolism of warfarin has 61 been studied very little. This is surprising because warfarin and especially the hydroxylated products are attractive substrates for phase II glucuronidation by UDP-glucuronosyltransferases (UGTs). Conjugation of xenobiotic compounds with glucuronic acid (GLUC) by UGTs is generally thought to be important for the transport and excretion of xenobiotic compounds through urine and/or bile (8). GLUC conjugation of warfarin metabolites is known to occur in rodents (9), and Kaminsky and Zhang (5) suggest that glucuronide metabolites of warfarin are excreted in human urine. Recent studies have begun characterizing the overall importance of this pathway by using human recombinant UGT expression systems (10, 11). These studies demonstrate that 6-, 7-, and 8-OH-WARs are glucuronidated by multiple UGTs including hepatic (UGT1A1, 1A3, and 1A9) and extra hepatic (UGT1A8 and 1A10) enzymes. Only UGT1A10 con jugates 4′-OH-WAR, and no known UGTs recognize WAR or 10-OH-WAR as substrates. The development of “omic” approaches reflects the desire to broaden the scope of efforts correlating biomarkers (genes, transcripts, proteins, or molecules) to pharmacological outcomes and thus improve our understanding of drug targeting and processing. Pharmacogenomics utilizes genomic information to understand individual variations in drug response. While early reports linked genes and warfarin treatment through familial resistance to drug treatment, recent multigene and genome-wide analyses indicate that CYP2C9 and vitamin K epoxide reductase (VKER) polymorphisms account for approximately 10 and 25%, respectively, of the population variance observed in warfarin dosage (12). Other genes are also implicated in these studies, although further work is necessary to confirm their significance. Only recently has a study broadened the scope of pharmaco genomic analysis to include potential phase II enzymes, which may clarify their significance in warfarin metabolism following further refinement of the statistical algorithm (13). Metabolomics is a comprehensive and quantitative analysis of metabolites in biological systems and thus more closely reflects the phenotype of the individual (14). Specifically, metabolic profiles of drugs reflect processing by multiple enzymes, whose activities are modulated by genetic polymor phisms, age, sex, diet, and the presence of other drugs and confounding factors. For warfarin, many of these factors are known to contribute to complex drug inactivation and clearance pathways for a particular individual. By utilizing the end products of metabolism for determining treatment strategy, metabolomics potentially offers a higher degree of personalization. As an initial step toward developing a metabolomic approach for warfarin treatment, a targeted method to identify and quantify phase I P450 and phase II UGT warfarin metabolites was developed. The chiral liquid chromatography-tandem mass spectrometry (LC-MS/MS) method developed has the unique ability to quantitatively measure warfarin, OH-WARS, and corresponding glucuronides in human urine while accounting for specific enantiomeric contributions. Three commercially available urine samples from patients undergoing warfarin therapy were used to test this new system and to begin simultaneously assessing the importance of multiple metabolic pathways involved in warfarin detoxification. Supporting evidence is provided, which shows that both phase I P450 and phase II UGT reactions are important for efficient warfarin detoxification and excretion. These analyses confirm the importance of CYP2C9 metabolism and reveals for the first time that glucuronides of warfarin and OH-WARs are formed in vivo at high levels.

### Materials and Methods

### Materials.

All chemicals used in this study were of at least reagent grade. Unless otherwise specified, all chemicals and reagents were purchased from either Sigma-Aldrich (St. Louis, Mo.) or Thermo Fisher Scientific (Waltham, Mass.). Racemic warfarin, 4′-OH-WAR, 6-OH-WAR, 7-OH-WAR, 8-OH-WAR, 10-OH-WAR, 7-hydroxycoumarin, as well as (R)-warfarin and (S)-warfarin were used as primary standards. Ethyl alcohol (100%) was purchased from AAPER (Shelbyville, Ky.). Re-combinant CYP2C9*1, CYP3A4, CYP1A2, and CYP2C19 were expressed in baculovirus-infected insect cells and purchased from BD Biosciences (San Jose, Calif.). Commercially available urine specimens from three females (F84, F85, and F87) were provided by the Emergency Response and Air Toxicants Branch, Division of Laboratory Science National Center for Environmental Health Centers for Disease Control and Prevention (DeKalb County, Ga.). Patient studies were conducted under IRB-approved protocols, and all patients signed informed consent documentation. Other than age, gender, and that each was receiving warfarin, no specific patient histories were provided.

### LC-MS/MS Analysis.

An Agilent 1100 HPLC system coupled to an API4000 Triple Quadrupole (MS/MS) mass spectrometer (Applied Biosystems) was used to analyze and quantify WAR, OH-WARS, and the corresponding glucu-ronides. Initial attempts to resolve all enantiomeric contributions in a single analysis proved unsuccessful. The (R)-isomers of 6-, 7-, and 8-hydroxywarfarin could not be separated; therefore, measurements of these metabolites were accomplished in two analytical phases. The first analytical phase fully resolved warfarin and the various hydroxywarfarins but did not account for specific enantiomers. This analytical phase was accomplished by reverse-phase HPLC using a 4.6 mm×150 mm C18 Zorbax Eclipse 5 μm XDB-C18 column provided by Agilent (Santa Clara, Calif.). The operating temperature was 40° C., and the sample injection volume was 5.0 μL. HPLC mobile phases consisted H₂O (0.1% acetic acid v/v) (A) and methanol (0.1% acetic acid v/v) (B). The flow rate was 1 mL/min. A gradient was used to elute warfarin species as follows: 50% A and 50% B for the first 30 s, followed by a linear gradient from 50 to 90% B (0.5-5.5 min), 90 to 100% B (5.5-5.6 min), 100% B maintained for 2.5 min, and 100 to 50% B (7.9-8.0 min) with 50% B maintained for 2 min. The total run time per sample was 10 min. Specific MS/MS experimental conditions used in this study are noted in Table 1.

TABLE 1
Positive Mode Multiple Reaction Monitoring (MRM) Experimental
Conditions Used for Diastereomer Assessments

| | | Analyte | Q1 (m/z) | Q3 (m/z) | CE (V) | EP (V) | DP (V) | CXP (V) |
|---|---|---|---|---|---|---|---|---|
| MRM | | WAR | 309.2 | 163.3 | 22 | 14 | 61 | 9 |
| | | WAR-O-GLUC | 485 | 163 | 45 | 10 | 52 | 17 |
| | 2 | 4′-OH-WAR | 325.3 | 267.1 | 29 | 10 | 53 | 15 |
| | | 4′-O-GLUC | 501.2 | 267.1 | 29 | 10 | 53 | 15 |
| | | 6-, 7-, and 8-OH-WAR | 325.3 | 179.2 | 23 | 10 | 53 | 10 |
| | | 6-, 7-, and 8-GLUC | 501.2 | 179.2 | 33.5 | 10 | 52 | 10 |
| | 3 | 10-OH-WAR | 325.3 | 251.2 | 28 | 10 | 53 | 17 |
| | | 10-O-GLUC | 501.3 | 251.2 | 31 | 10 | 52 | 14 |
| | 4 | 7-hydroxycoumarin | 163 | 107 | 31 | 10 | 122 | 6 |

MS/MS analyses were performed in positive ion mode by electrospray ionization (ESI) using a Turbo IonSpray source. Curtain, nebulizer, turbo, and collisionally-activated dissociation gases were 40 psig, 50 psig, 65 psig, and 6 psig, respectively. Turbo heater temperature was 510° C., and ion spray voltage was 5500 V.CE=Collision Energy, EP=Entrance Potential, DP=Declustering Potential, and CXP=collision Cell Exit Potential

To determine the relative abundance of the (R)-and (S)-enantiomers of each metabolite, a 4.6 mm×25 cm Chirobiotic V5 μm column supplied by Supelco (Bellefonte, Pa.) was incorporated during the second analytical phase. Mobile phases consisted of H₂O (0.1% acetic acid v/v) (A) and acetonitrile (0.1% acetic acid v/v) (B) with a flow rate of 800 μL/min. Enantiomeric separations were accomplished under isocratic conditions (80% A and 20% B). The sample injection volume was 15.0 μL. Enantiomeric MS/MS analyses were performed in negative ion mode by electrospray ionization (ESI) using a Turbo IonSpray source. A total run time of 20 min was required for adequate resolution. Specific MS/MS experimental conditions used in this study are noted in the Table 2.

TABLE 2
Negative Mode Multiple Reaction Monitoring (MRM) Experimental
Conditions Used for Enantomeric Assessments

| | | | Q1 | Q3 | | | | |
|---|---|---|---|---|---|---|---|---|
| | | Analyte | (m/z) | (m/z) | CE (V) | EP (V) | DP (V) | CXP (V) |
| MRM | 1 | WAR | 307 | 161 | −26 | −30 | −62 | −14 |
| | 2 | 6-, 7-, and 8-OH- | 323 | 177 | −30 | −7 | −70 | −15 |
| | | WAR | | | | | | |
| | 3 | 10-OH-WAR | 323 | 251 | −26 | −10 | −50 | −15 |
| | 4 | 4′-OH-WAR | 323 | 145 | −48 | −10 | −55 | −10 |
| | 5 | 7-hydroxycoumarin | 161 | 65 | −44 | −10 | −70 | −9 |

MS/MS analyses were performed in negative ion mode by electrospray ionization (ESI) using a Turbo IonSpray source. Curtain, nebulizer, turbo, and collisionally-activated dissociation gases were 40 psig, 70 psig, 70 psig, and 6 psig, respectively. Turbo heater temperature was 550° C., and ion spray voltage was −4500 V.CE=Collision Energy, EP=Entrance Potential, DP=Declustering Potential, and CXP=collision Cell Exit Potential

### Quantitative Measurements.

An internal standard method was used to quantitatively measure warfarin and OH-WARS by comparing relative MRM responses associated with authentic standards (0.5-100 nM) made in human base urine known to be free of warfarin and warfarin metabolites. Each response was normalized to that of the internal standard, 7-hydroxycoumarin (10 μM). When urine was treated with an excess of GUS, GLUC-conjugated warfarin metabolites were liberated, allowing quantitative measurements of the corresponding OH-WARs and/or warfarin. Enantiomeric determinations of (R)-WAR, (R)-4′194 OH-WAR, (R)-10-OH-WAR, as well as (S)-WAR and all of the (S)-OH-WAR metabolites were directly derived from standard curves generated with authentic standards. However, (R)-6-, (R)-7-, and (R)-8-OH-WAR coeluted and could not be measured directly. Measurements of these three metabolites were calculated by taking the difference of the amount measured in the first analytical phase, which contained both (R)-and (S) isomers from the amount of the (S)-isomer measured in the second analytical phase (eq 3). Glucuronidated metabolites were measured by incubating 250 μL of urine with 2000 units of GUS in 100 mM sodium acetate buffer, pH 5.0. The final volume for each of these reactions was 1 mL. Control reactions omitting GUS were included with each sample set. Reactions were incubated overnight at 37° C., after which they were stored at −80° C. Immediately before analysis, both samples were diluted with an equal volume of ethanol containing 7-hydroxycoumarin (10 μM final concentration). The difference between GUS-treated samples and controls represented the amount of glucuronidated metabolites present. Example calculations are as follows:

### Example Calculation of Glucuronides.

[7-OH-WAR]_{GUS}−[7-OH-WAR]_{control})=[7-O-GLUC] (1)

### Example Calculation of Glucuronide Enantiomers.

[(S)-7-OH-WAR]_{GUS}−[(S)-7-OH-WAR]_{control})=[(S)-7-O-GLUC] (2)

### Example Calculation of (R)-Glucuronide Enantiomers.

[7-O-GLUC]_{measured in analytical phase I}−[(S)-7-O-GLUC]_{measured in analytical phase II})=[(R)-7-O-GLUC] (3)

### Precision, Accuracy, and Method Detection Limit Studies.

Quality control high (100 nM) and quality control low (10 nM) solutions were made from base human urine. Quality control samples were spiked with a mixture of WAR, 4′-OH-WAR, 6-OH-WAR, 7-OH-WAR, 8-OH-WAR, and 10-OH-WAR. Because WAR and OH-WARs exist as racemic mixtures, the same quality control high and quality control low solutions were used for enantiomeric measurements, but concentrations of each enantiomer were 50 and 5 nM, respectively. Accuracy and precision were evaluated over nonconsecutive days by taking the mean of seven independent analyses and calculating the % RSD. The lower limits of quantitation for each analytical phase were determined by multiplying the standard deviation of the quality control low sample by 3. Recombinant Cytochrome P450 Incubations and GUS Reactions. Commercially available enantiomers of WAR were used to make enantiomeric assignments. Enantiomeric pure OH-WARs were generated through specific chemoenzymatic reactions. Cytochrome P450s 1A2, 3A4, 2C19, and 2C9*1 were each incubated individually with (R)-WAR or (S)-WAR. The enzyme was at 25 nM in 50 μM KPi buffer. The substrate was at 1 mM final concentration with EDTA at a final concentration of 0.1 mM. A glucose-6-phosphate NADPH regeneration system was used with NADP+, G6P, and G6PDH in excess. Each reaction was incubated for 30 min at 37° C. and quenched with an equal volume of ethanol containing 7-hydroxycoumarin for a final concentration of 10 μM. These reactions generated stereospecific OH-WARs, which could be used for OH-WARs enantiomeric retention time assignments.

### Results

### Analytical Method Development and Validation.

Separation was optimized for the measurement of warfarin and monohydroxylated warfarin derivatives during the first analytical phase. Internal standard (7-hydroxycoumarin), WAR, 4′-, 6-, 7-, 8-, and 10-OH-WAR achieved baseline separation. Retention times for 7-hydroxycoumarin, WAR, 4′-, 6-, 7-, 8-, and 10-OH-WAR were 2.7, 6.6, 5.0, 5.6, 6.0, 6.2, and 5.4 min, respectively (FIG. 2A). Standard curves (0.5-100 nM) provided a wide linear working range (r²≧0.999), and recovery of quality control specimens at 10 and 100 nM showed a high degree of precision and accuracy (Table 3). Similar results were also observed during the second analytical phase. A chiral LC-MS/MS method was optimized for separation and measurement of specific warfarin enantiomers. Internal standard (7-hydroxy-coumarin), (S)-WAR, (S)-4′-, (S)-6-, (S)-7-, (S)-8-, and (S)-10-OH-WAR achieved baseline separation (FIG. 2B). Retention times for 7-hydroxycoumarin, (S)-WAR, (S)-4′-, (S)-6-, (S)-7-, (S)-8-, and (S)-10-OH-WAR were 6.8, 16.5, 13.9, 13.5, 14.5, 12, and 11.2 min, respectively. (R)-WAR, (R)-4′-OH-WAR, and (R)-10-OH-WAR eluted at approximately 12, 9.5, and 8 min, respectively, while three enantiomers, (R)-6-, (R)-7-, and (R)-8-OH-WAR, coeluted around 10 min. Standard curves (0.25-50 nM) provided a wide linear working range (r²≧0.991). Recovery of quality control specimens at 5 and 50 nM showed a high degree of precision and accuracy (Table 3). For both analytical phases, MDLs and LLQs are provided for each analyte (Table 3). MRLs were 8-fold higher than the LLQ to incorporate dilution factors associated with GUS treatment. All metabolites were quantitatively measured in each analytical phase as described in the Materials and Methods.

TABLE 3
Summary of Correlation Coefficients, Quality Control
Measurements, and Minimum Detection and Reporting Limits^{a}

| | correlation | mean (% RSDb | | | | |
|---|---|---|---|---|---|---|
| | coefficient | quality control | quality control | nM | | |
| phase I | (R2) | low (10 nM) | high (100 nM) | MDLc | LLQe | MRLd |
| 4′-OH-WAR | 0.999 | 10.6 (23 (33) | 104 (17 (24) | <0.5 | 7 | 59 |
| 10-OH-WAR | 0.999 | 9.4 (31 (44) | 102 (19 (27) | <0.5 | 9 | 70 |
| 6-OH-WAR | 0.999 | 10.3 (21 (30) | 101 (22 (31) | <0.5 | 7 | 53 |
| 7-OH-WAR | 0.999 | 10.7 (25 (35) | 102 (19 (27) | <0.5 | 8 | 65 |
| 8-OH-WAR | 0.999 | 11.1 (16 (23) | 105 (15 (21) | <0.5 | 5 | 43 |
| WAR | 0.999 | 10.4 (23 (33) | 105 (14 (20) | <0.5 | 7 | 58 |
| | correlation | mean (% RSDb | | | | |
| | coefficient | quality control | quality control | nM | | |
| phase II | (R2) | low (5 nM) | high (50 nM) | MDL | LLQ | MRL |
| (R)-WAR | 0.999 | 5.5 (16 (23) | 46 (22 (31) | <0.25 | 3 | 21 |
| (S)-WAR | 0.998 | 5.6 (12 (17) | 55 (11 (16) | <0.25 | 2 | 17 |
| (S)-4′-OH- | 0.994 | 5.4 (24 (34) | 40 (36 (51) | <0.25 | 4 | 31 |
| WAR | | | | | | |
| (S)-10-OH- | 0.994 | 5.1 (51 (72) | 45 (14 (20) | <0.25 | 8 | 64 |
| WAR | | | | | | |
| (S)-6-OH- | 0.994 | 5.3 (20 (28) | 46 (17 (24) | <0.25 | 3 | 26 |
| WAR | | | | | | |
| (S)-7-OH- | 0.998 | 5.3 (22 (31) | 45 (24 (34) | <0.25 | 4 | 28 |
| WAR | | | | | | |
| (S)-8-OH- | 0.999 | 5.5 (15 (21) | 51 (27 (38) | <0.25 | 3 | 20 |
| WAR | | | | | | |
| (R)-4′-OH- | 0.991 | 5.4 (17 (24) | 48 (17 (24) | <0.25 | 3 | 21 |
| WAR | | | | | | |
| (R)-10-OH- | 0.991 | 5.3 (19 (27) | 53 (18 (25) | <0.25 | 3 | 25 |
| WAR | | | | | | |

^{a}Values in parentheses represent the errors associated with calculations used to determine glucuronide concentrations. The errors associated with calculations used to determine (R)-6-, (R)-7-, and (R)-8-OHWAR concentrations ranges were 29, 33, and 22% RSD, respectively.
b% RSD) 100(CV/mean).
cMDL) minimum detection limit.
dMRL) minimum reporting limit.
eLLQ) lower limit of quantification.

### Urine Sample Analysis.

In an effort to assess the applicability of this new method and to begin assessing the toxicological significance of metabolic pathways now being recognized as important determinants for warfarin toxicity (10, 11), WAR and OH-WARs were measured in three purchased human urine samples received from female donors. Chromatographs obtained for F84 are provided as representative examples (FIGS. 1E, F and 2B, C). Retention times and specific mass transitions of urinary metabolites corresponded well with authentic standards (FIG. 2A, D). Optimization parameters previously established for OH-WAR-GLUCs (10) were included during the first analytical phase to ensure that GUS treatment was complete. The complete disappearance of glucuronides and a concomitant increase in WAR and OH-WAR responses post-GUS treatment shows complete GLUC hydrolysis (FIG. 2B, C). Relative abundances of all of the WAR metabolites measured were highly variable among the specimens (FIG. 3). Both native WAR and 4-O-GLUC-WAR (glucuronide of native warfarin) were identified in each of the three urinary samples, but direct WAR glucuronidation seemed to be primarily associated with the (R)-enantiomer (FIG. 3). The most predominate metabolites observed in all three specimens were 6-OH-WAR, 7-OH-WAR, and their corresponding GLUC conjugates (FIG. 3). Lower concentrations of 4′-OH-WAR, 8-OH-WAR, and all of the corresponding GLUC metabolites were also detected in these specimens (FIG. 3). 10-OH-WAR was not detected in any specimen (FIG. 3). Mass spectra for the glucuronidated metabolites presented in this report have previously been reported (10).

The total amount of OH-WAR excreted (free and conjugated) in these urine specimens ranged from approximately 1to 4 μM (Table 4). These high concentrations demonstrate that both phase I and II metabolic pathways are highly active in the presence of WAR and OH-WARs. The predominance of (S)-6-and (S)-7-OH-WAR in all three specimens suggests significant CYP2C9 activity (4), while the combined presence of other OH-WARs suggests the involvement of other P450s. WAR, 4′-OH-WAR, 8-OH-WAR, and corresponding GLUC metabolites were found at lower individual concentrations (10-200 nM); however, the combined contribution of all of these metabolites represents multiple detoxification pathways, which when evaluated together demonstrate a large human capacity for detoxifying and excreting warfarin. It is also interesting to note the seemingly enantioselective reactions observed for both phase I and II reactions (FIG. 3 and Table 4). For example, it appears that (S)-7-OH-WAR is selectively glucuronidated (FIG. 3A, B), although some patients appear to have the ability to glucuronidate both (S)- and (R)-7-OHWAR (FIG. 3C).

TABLE 4
Total P450 Metabolism/Contribution Summary^{a}

| | | 6-OH-WAR | 7-OH-WAR | 8-OH-WAR | 10-OH-WAR | 4′-OH-WAR | total |
|---|---|---|---|---|---|---|---|
| F84 | urinary concentration (nM) | 560 | 1964 | 111 | ND | 140 | 2775 |
| | % conjugated % S | 35 35 | 69 20 | 31 0 | ND ND | 47 56 | 58 77 |
| F85 | % R | 65 | 80 | 100 | ND | 64 | 33 |
| | urinary concentration (nM) | 1124 | 2404 | 109 | ND | 94 | 3731 |
| | % conjugated % S | 15 31 | 28 62 | 53 2 | ND ND | ND 75 | 27 70 |
| F87 | % R | 69 | 38 | 98 | ND | 25 | 30 |
| | urinary concentration (nM) | 204 | 724 | 231 | ND | 20 | 1179 |
| | % conjugated % S | 12 38 | 14 48 | 7 31 | ND ND | ND 70 | 14 47 |
| | % R | 62 | 52 | 69 | ND | 30 | 53 |

^{a}The % S represents the proportion of each metabolite that is in the (S)-enantiomeric form. The % conjugated represents the proportion of each phase I metabolite that is glucuronidated.
ND, not detected.
The error associated with these values is described in Table 3.

Data presented also show the importance of considering both phase I and II metabolic pathways while assessing specific P450 contributions. For example, the total amount of glucuronides varied significantly among the three specimens (14-58%), which can cause miscalculation of P450 product ratios if UGT metabolism is ignored. The predicted product ratio of (S)-6337 OH-WAR:(S)-7-OH-WAR is approximately 0.2 (4), which is the case for F87 and F88. The product ratio for F84 is approximately 0.5 when GLUC metabolites are ignored but 0.2 340 when included. By assessing both P450 and UGT metabolites, all three patients are normalized, and resulting data suggest that (S)-6-OH-WAR and (S)-7-OH-WAR formation are primarily mediated through CYP2C9 catalysis.

### Discussion

The LC-MS/MS approach developed in this study has the unique ability to identify and measure 24 WAR metabolites in human urine. This is the first in vivo report to demonstrate that humans have a high capacity to directly glucuronidate warfarin and its P450-generated metabolites. High concentrations of glucuronides demonstrate that UGT phase II metabolism is very active in humans and represents an important pathway for efficient warfarin detoxification and excretion. The predominance of (S)-7-OH-WAR is consistent with the widely acknowledged significance of CYP2C9 in WAR metabolism (15), but other “minor” P450 pathways collectively contribute as much or more than CYP2C9 to the generation of excretable metabolites. (S)-7-OH-WAR, a marker metabolite for CYP2C9 (14), is the most abundant (S)-WAR P450 metabolite identified in these studies (61-71%). CYP2C9 also generates (S)-6-OH-WAR but with 4-fold less efficiency (4, 16). Product ratio analyses show that CYP2C9 alone accounts for approximately 78-95% of all (S)-WAR metabolites. The identities and levels of remaining (S)-WAR metabolites are highly variable. (S)-8-OH-WAR accounts for 4-20% of the metabolites measured. CYP3A4 is thought to produce this metabolite (4), but the absence of 10366 OH-WAR argues against this metabolic pathway. (S)-4′-OH367 WAR is then probably derived from CYP2C8, CYP2C18, and/or CYP2C19 activity (17). Limited kinetic investigations with (R)-warfarin make as signing P450 contribution for this isomer difficult. The production of (R)-8-OH-WAR in F85 and F87 shows the significance of CYP2C19 in warfarin metabolism (18), although CYP1A2 also produces this metabolite (7). CYP1A2 specifically generates (R)-6-and (R)-8-OH-WAR at a ratio of 5:1. Increased production of (R)-6-OH-WAR in F85 is suggestive of CYP1A2 induction, possibly through tobacco smoke exposures. The major (R)-metabolite for F84 and F87 is (R)-7-OH-WAR, which is possibly a marker product for CYP2C8 activity (17). As observed for the (S)-isomer, there are no measurable levels of (R)-10-OH-WAR, and low levels of (R)-4′-OH-WAR may indicate CYP2C8 and/or CYP2C18 activity (17). Glucuronidation presumably promotes the elimination of metabolites through selective transporters and abolishes any VKER binding due to the introduction of a bulky acidic sugar. The 4-hydroxyl group of WAR, the only site available for conjugation, is required for VKER binding (19, 20). The preferential glucuronidation of the (R)-isomer among the patients may partly explain the lower biological activity of this form of the drug (3), although it is not known whether (R)-or (S)-WAR bind VKER with differing affinities. WAR oxidation by P450s introduces a second hydroxyl group for conjugation, resulting in more efficient glucuronidation (10). The diversity and levels of glucuronides in urine attest to the significance of UGT activity toward these compounds. Assigning specific UGT contributions at this time is difficult because only racemic mixtures have been used in vitro to assess specific activities and because the isozyme responsible for direct warfarin glucuronidation has not been identified (10). However, some conclusions can be made from human data presented in this report. Production of 6-O-GLUC is indicative of UGT1A1 and/or UGT1A10 activity (10), and the contribution of two enzymes is further evidenced in this report by the preferential excretion of the (S)-6-O-GLUC isomer in F84 and the (R)-6-O-GLUC isomer in F85 and F87. The glucuronidation of 7-and 8-OH-WARs is more complex due to the potential involvement of UGT1A1, UGT1A3, UGT1A8, UGT1A9, and/or UGT1A10 (10). The presence of 4′-O-GLUC is indicative of UGT1A10 activity (10).

Human data presented in this report begin demonstrating the complexity of warfarin detoxification and begin characterizing the significance of both phase I and II enzymes. The significance of phase I metabolism has been the focus of much research for many years, but phase II glucuronidation is just now being recognized as another indispensible step for warfarin detoxification. Little focus has been given to hydroxywarfarin glucuronidation because hydroxywarfarins are generally thought to be biologically inactive. However, early rodent experiments conducted in Karl Link's laboratory show that hydroxywarfarins are fatal to rats receiving 6 mg/kg (21). The significance of this study is limited because only a single dose is used and there is little to no repetition. Nevertheless, recent VKER studies demonstrate that 6-OH-WAR retains similar binding affinity toward the drug target as shown for biologically active warfarin alcohols (20), and it seems that 4′-modifications of warfarin increase affinity toward VKER (20) and anticoagulant efficacy (22). It was also shown that hydroxywarfarins can inhibit P450s in vitro at toxicologically relevant concentrations through a negative feedback mechanism (10). Continued characterization of these metabolic pathways may improve the ability to fully assess the physiological significance of phase I and II warfarin metabolites.

The widespread usage and efficacy of WAR is driving many efforts aimed at developing approaches that improve drug treatment strategies and consequently pharmacological outcomes. This report validates a comprehensive LC-MS/MS method capable of assessing P450 and UGT contribution to warfarin metabolism. Human data confirm that OH-WARs reach high enough concentrations in vivo to serve as substrates for UGTs. Furthermore, it appears that glucuronidation is a major pathway of WAR detoxification and excretion by either direct warfarin conjugation or through subsequent metabolism of OH-WARs. Taken together, both P450 and UGT metabolic pathways are important for facilitating the flux of warfarin through complex metabolic pathways.

### REFERENCES FOR EXAMPLE 1

1. Lesko, L. (2008) The critical path of warfarin dosing: finding an optimal dosing strategy using pharmacogenetics. Clin. Pharmacol. Ther. 84, 301-303.
2. Bristol-Myers (2005) Coumadin tablets.
3. Eble, J. N., West, B. D., and Link, K. P. (1966) A comparison of the isomers of warfarin. Biochem. Pharmacol. 15, 1003-1006.
4. Rettie, A. E., Korzekwa, K. R., Kunze, K. L., Lawrence, R. F., Eddy, C., Aoyama, T., Gelboin, H. V., Gonzalez, F. J., and Trager, W. F. (1992) Hydroxylation of warfarin by human cDNA-expressed cyto chrome P-450: A role for P-4502C9 in the etiology of (S)-warfarin drug interactions. Chem. Res. Toxicol. 5, 54-59.
5. Kaminsky, L. S., and Zhang, Z. Y. (1997) Human P450 metabolism of warfarin. Pharmacol. Ther. 73, 67-74.
6. Wadelius, M., Chen, L. Y., Eriksson, N., Bumpstead, S., Ghori, J., Wadelius, C., Bentley, D., McGinnis, R., and Deloukas, P. (2007) Association of warfarin dose with genes involved in its action and metabolism. Hum. Genet. 121, 23-34.
7. Zhang, Z., Fasco, M. J., Huang, Z., Guengerich, F. P., and Kaminsky, L. S. (1995) Human cytochromes P450 1A1 and 1A2: R-warfarin as a probe. Drug Metab. Dispos. 23, 1339-1345.
8. Ritter, J. K. (2000) Roles of glucuronidation and UDP-glucuronosyl transferases in xenobiotic bioactivation reactions. Chem.-Biol. Interact. 129, 171-193.
9. Jansing, R., Chao, E., and Kaminsky, L. (1992) Phase II metabolism of warfarin in primary culture of adult rat hepatocytes. Mol. Pharmacol. 41, 209-215.
10. Zielinska, A., Lichti, C. F., Bratton, S., Mitchell, N. C., Gallus Zawada, A., Le, V.-H., Finel, M., Miller, G. P., Radominska Pandya, A., and Moran, J. H. (2008) Glucuronidation of monohy droxylated warfarin metabolites by human liver microsomes and human recombinant UDP-glucuronosyltransferases. J. Pharmacol. Exp. Ther. 324, 139-148.
11. Miller, G. P., Lichti, C. F., Zielinska, A. K., Mazur, A., Bratton, S. M., Gallus-Zawada, A., Finel, M., Moran, J. H., and Radominska Pandya, A. (2008) Identification of hydroxywarfarin binding site in human UDP glucuronosyltransferase 1A10: Phenylalanine90 is crucial for the glucuronidation of 6-and 7-hydroxywarfarin but not 8-hydroxywarfarin. Drug Metab. Dispos. 36, 2211-2218.
12. Au, N., and Rettie, A. E. (2008) Pharmacogenomics of 4-hydroxy coumarin anticoagulants. Drug Metab. ReV.40, 355-375.
13. Cooper, G. M., Johnson, J. A., Langaee, T. Y., Feng, H., Stanaway, B., Schwarz, U. I., Ritchie, M. D., Stein, C. M., Roden, D. M., Smith, J. D., Veenstra, D. L., Rettie, A. E., and Rieder, M. J. (2008) A genome-wide scan for common genetic variants with a large influence on warfarin maintenance dose. Blood 112, 1022-1027.
14. Dettmer, K., Aronov, P. A., and Hammock, B. D. (2007) Mass spectrometry-based metabolomics. Mass Spectrom. ReV.26, 51-78.
15. Rettie, A., Korzekwa, K. R., Kunze, K. L., Lawrence, R. F., Eddy, C., Aoyama, T., Gelboin, H. V., Gonzalez, F. J., and Trager, W. F. (1992) Hydroxylation of warfarin by human cDNA-expressed cytochrome P-450: A role for P-4502C9 in the etiology of (S)-warfarin-drug interactions. Chem. Res. Toxicol. 5, 54-59.
16. Haining, R. L., Hunter, A. P., Veronese, M. E., Trager, W. F., and Rettie, A. E. (1996) Allelic variants of human cytochrome P450 2C9: Baculovirus-mediated expression, purification, structural characterization, substrate stereoselectivity, and prochiral selectivity of the wild-type and I359L mutant forms. Arch. Biochem. Biophys. 333, 447-458.
17. Kaminsky, L., de Morais, S., Faletto, M., Dunbar, D., and Goldstein, J. (1993) Correlation of human cytochrome P4502C substrate specificities with primary structure: Warfarin as a probe. Mol. Pharmacol. 43, 234-239.
18. Wienkers, L. C., Wurden, C. J., Storch, E., Kunze, K. L., Rettie, A. E., and Trager, W. F. (1996) Formation of (R)-8-hydroxywarfarin in human liver microsomes: A new metabolic marker for the (S)-mephenytoin hydroxylase, P4502C19. Drug Metab. Dispos. 24, 610-614.
19. Lee, J. J., and Fasco, M. J. (1984) Metabolism of vitamin K and vitamin K 2,3-epoxide via interaction with common disulfide. Biochemistry 23, 2246-2252.
20. Gebauer, M. (2007) Synthesis and structure-activity relationships of novel warfarin derivatives. Bioorg. Med. Chem. 15, 2414-2420.
21. Eble, J. N. (1953) Studies on the 4-hydroxycoumarin anticoagulants warfarin and cyclocumarol. University of Wisconsin Library, Part II, pp 22-30.
22. Lasseur, R., Grandemange, A., Longin-Sauvageon, C., Berny, P., and Benoit, E. (2007) Comparison of the inhibition effect of different anticoagulants on vitamin K epoxide reductase activity from warfarin susceptible and resistant rat. Pestic. Biochem. Physiol. 88, 203-208.

### Example 2

Every year, two million people begin Coumadin (R-, S-Warfarin [War]) therapy in the United States. War is a challenging drug to accurately dose, both initially and for maintenance, because of its narrow therapeutic range, wide inter-patient variability, and long list of factors that can influence dosing. Recently, War was reported to be the fourth leading cause of adverse drug events. Therefore, the development of improved treatment and effective management in anticoagulant therapy strategies remains an important challenge. It is well established that effective personalization of War therapy may have to rely on much more than just pharmacogenomic information. The current challenge for personalizing War therapy is to develop better clinical diagnostics that can account for all confounding factors and develop better anticoagulant drugs by improving the understanding of War metabolic pathways. Due to the limitations of the traditional approach, personalizing War therapy is limited, and emphasizes pharmacogenomics, which utilizes genetic information related to polymorphisms of cytochrome P450 (CYP)2C9 and VKORC1 to understand better individual variations in drug response. However, these underlying genetic factors have been shown to account for only ˜35-40% of variations in patient response to War, which leaves significant room for improvement. As an alternative to genotyping, the strategy of metabolic phenotyping of patients is being pursued using a metabolomics approach. It is anticipated that patient metabolic profiles, which also include War conjugates such as glucuronides and sulfates may yield biomarkers that correlate with dose response and relative levels of metabolites as signatures for contributions of specific enzymes to the inactivation and elimination of War.

One of the most important deficiencies in the evaluation of War dosing was lack of understanding of the role of Phase II enzymes (UDP-glucuronosyltransferases [UGTs] and sulfotransferases [SULTs]) in War metabolism. These previously ignored pathways and the enzymes involved can contribute significantly to the total metabolic profile for War biotransformation and potentially yield significant undiscovered biomarkers. Based on the important discoveries in this area, it was hypothesized that full metabolic profiles of War from urine and/or plasma may represent a unique signature of the specific contributions of Phase I and II enzymes to War biotransformation. It was further hypothesized that biomarkers present in these metabolic profiles may reflect the capacity of individuals to inactivate and eliminate War and thus affect the drug treatment strategy.

The long-term goal of this project is to minimize the complications of War therapy by using War metabolites as biomarkers for its inactivation and clearance. Based on the discovery of the significance of Phase II War metabolism, it was proposed to focus on the identification and quantitation all Phase I and II War metabolites, which may provide the basis for translating metabolic profiles into a biological readout of each patient's metabolic capacity. As a first step toward exploring this metabolomic approach to personalized War therapy, current high pressure liquid chromatography—tandem mass spectrometry (LC-MS/MS) techniques was expanded on to quantify War and corresponding hydroxyWars (OHWars), glucuronides (-Glucs), and sulfates, generated by CYPs, UGTs, and SULTs, respectively. This method accounts for specific enantiomeric contributions, which are important for understanding War metabolism. In total, the LC-MS/MS procedure can simultaneously measure over 36 hydroxylated, glucuronidated and sulfated War enantiomers. The most unique qualities of this procedure are its simplicity, sensitivity, accuracy, precision, and reliability.

The existing research related to the identification of War metabolites in humans is very limited, mainly because a ‘good tool’ has not been available to measure these compounds in human specimens. Although several HPLC and LC-MS/MS studies have been reported, those efforts were limited to the determination of War and its hydroxylated metabolites in urine and plasma, and excluded the analyses of Phase II conjugation products. This selective analysis of the War metabolome is a significant weakness, which limits the ability to understand fully War detoxification pathways. The in vivo preliminary data identified War glucuronides in human urine and demonstrated that in some cases the percentage of War metabolites recovered in urine as glucuronides can represent up to 60% of all the War metabolites detected. Moreover, our in vitro experiments show that OHWars are excellent substrates for glucuronidation. It is widely recognized that glucuronidation, which significantly increases the polarity of the conjugated products over that of the parent compound, is an indispensable step for the excretion of many hydrophobic compounds. This process also incorporates a negative charge, which is recognized by various transporters (MRP2: Multidrug Resistance-Associated Protein and MDR: Multidrug Resistance Protein), into the molecule allowing for efficient, structure-independent export from the cell. More recently, this technology was expanded to include sulfated metabolites of War.

This research has an extremely broad potential to affect both basic research science and public health. Each year, about 2 million people join the over 50 million patients currently on War therapy for the short- and long-term management of thromboembolic and hemostatic disorders, such as deep-vein thrombosis, pulmonary embolism, inherited thrombophilia, and antiphospholipid syndrome. Physicians also prescribe War for a number of other medical conditions and treatments, including the prevention of myocardial infarction and stroke, atrial fibrillation, and orthopedic surgery. When used appropriately, War is a highly effective, safe, and inexpensive medication. Nevertheless, complications in treatment lead to as many as 43,000 patients experience bleeding complications each year that require emergency treatment. Because of these complications, in 1995 the Agency for Healthcare Policy and Research (AHCPR) reported that War is greatly underutilized for stroke prevention. This shows that physicians are reluctant to prescribe War fearing that the drug may cause life-threatening complications. The knowledge gained from this proposed research may provide critical guidance during War treatment to minimize the time necessary to optimize dosage, decrease treatment costs, and potentially avoid harmful drug-drug interactions and bleeding episodes that often require costly emergency room visits.

These discoveries and techniques have provides a unique position to contribute to the growing field of personalized medicine in which not only a patient's genotype, but also a comprehensive metabolic signature can be the basis for the selection of appropriate dosage for War therapy and monitoring its effectiveness. The metabolomics approach being discussed in this proposal, which also includes conjugation products of War, is a comprehensive and quantitative analysis of metabolites in biological systems and is indicative of the current metabolic capacity of the patient. This approach combines non-genetic and genetic factors representing all the enzymes involved, and takes into account not only differences associated with ethnicity/race, genetic polymorphisms, age, and gender, but also diet, concurrent drugs and confounding factors. These individual variations need to be incorporated into dosing algorithms for patients receiving War therapy. It only takes minutes to generate a metabolic profile and begin developing readouts of patient metabolic capacities.

The main focus of this research is to work toward minimizing complications of War therapy by using War metabolites as biomarkers for its inactivation and clearance. Patient capacity to metabolize War ultimately affects the required dosage to maintain an effective steady-state concentration of the drug. The identification and quantitation of all Phase I and II War metabolites may provide the basis for translating metabolic profiles into a biological readout of each patient's metabolic capacity.

Throughout this study the methods and technologies developed for this project may be further improved, the methods consisting of: 1) LC-MS/MS analysis of metabolites in urine and plasma, 2) semi-preparative HPLC separation of R and S enantiomers of OHWars from purchased and synthesized racemic mixtures, 3) biochemically and chemically synthesize glucuronide and sulfate reference standards for LC-MS/MS, analyses, and 4) synthesize deuterium labeled War conjugate reference standards. It is anticipated that during the course of this proposal it may be possible to provide enough information to begin in silico validation.

While many drugs may have only a few metabolites, racemic War is processed in humans to form more than 36 metabolites. War, which is composed of a racemic mixture of R and S enantiomers, is oxidized by multiple hepatic CYPs at 5 different positions to form 10 different OHWars as shown in FIG. 4. These OHWars can be conjugated further with UDP-GlcUA by UGTs to form 10 different glucuronides. Additionally, it has been revealed that War can be directly glucuronidated via the native hydroxyl at the 4 position. There is also preliminary evidence that sulfates of OHWars are also biosynthesized in vivo.

Recent studies have demonstrated that specific human hepatic and extrahepatic UGT isoforms are responsible for conjugating War and OHWars. Glucuronidation activity of human liver and human intestinal microsomes (HLM and HIM) and eight human recombinant UGTs towards (R)- and (S)-War, racemic War, and major CYP metabolites of War (4′-, 6-, 7-, 8-, and 10-OHWar) has been assessed. Although the in vitro work has not identified the UGT responsible for native War glucuronidation, this metabolite has been detected in human urine. This was the first study identifying and characterizing specific human UGT isozymes, which glucuronidate major CYP metabolites of War with similar metabolic rates known to be associated with War metabolism. This was also the first demonstration of the significant role of exclusively extrahepatic intestinal UGTs (UGT1A10 and 1A8) in the first pass clearance of War metabolites. Continued characterization of these pathways with different War enantiomers may enhance our ability to understand the causes of life-threatening and costly complications associated with racemic War therapy.

Therefore, the goal is to identify and characterize the human UGTs involved in the metabolism of R- and S-OHWar. First, all available UGT1A isoforms and UGT2B7, as a representative isoform from the 2B family, may be investigated for their ability to produce War glucuronides of each enantiomer. UGT1A10, 1A1, and 1A3 may be the primary focus of this aim due to the demonstration of the significance of these enzymes in glucuronidating OHWars. FIG. 4 shows a representative scheme indicating the specificity of the UGTs for OHWars and the corresponding sites of modification. LC-MS/MS methods have enabled the analysis of the product mixtures from reactions with recombinant UGTs, and corresponding OHWar-Glucs have been biosynthesized for use as standards for LC-MS/MS analysis.

### Experimental Approach

### Determine Activity of UGTs Toward R- and S-OHWars.

All available UGT isoforms (with an emphasis on those expressed in liver and intestine) may be screened for the glucuronidation of separated R and S enantiomers of each OHWar, using the same strategy described for the racemic mixtures. R and S enantiomers of OHWars may be separated by semi-preparative HPLC as described below and used as substrates. Steady state kinetic analysis then may be used to determine which of the UGT isoforms is most active and has the highest affinity toward each individual enantiomer as reflected by Vmax and Km values, respectively. Based on the results of our studies with racemic mixtures, it is reasonable to assume that multiple enzymes may be found to catalyze each enantiomer. In this circumstance, the most active UGT and any other isoform with at least 10% of that activity may be chosen for further study. Quantitation of glucuronide products may be based on the sensitivity to β-glucuronidase treatment, comparison to negative controls with UDP-GlcUA excluded from the incubation, and LC-MS/MS analysis.

### Compare Kinetic Parameters (Km and Vmax) Between Individual UGT Isoforms and Human Hepatic and Intestinal Microsomes.

The characterization of hepatic and extrahepatic glucuronidation may be made based on glucuronidation studies with 100 commercially available HLM and 100 intestinal microsomal preparations available in our laboratory. Preliminary studies have been done using racemic OHWars and selected UGT1A isoforms (Table 5). Both racemic mixtures and separated R and S enantiomers may be assayed. Steady state kinetic analysis may be then used to determine which of the most active UGT isoforms have Km values most similar to Km values for the HLM and HIM. Activities of extrahepatic (UGT1A8 and UGT1A10) and hepatic (UGT1A1, UGT1A3, and UGT1A9) isoforms may be compared to that of their respective microsomal preparations, and assign the contribution of specific UGTs to product formation by the human samples. The use of two substrate concentrations (at about the Km value and 10 times the Km for the HLM and HIM) for screening may also assist in identifying low affinity UGTs that are less likely to be active toward substrates at clinically relevant concentrations.

TABLE 5
Steady-state parameters for glucuronidation of warfarin
metabolites by UGT isoforms. Kₘ is given in μM and Vₘₐₓ is
given in nmol/mg protein/min.

| | Km | Vmax |
|---|---|---|
| 6-OH-Warfarin | | |
| UGT1A10 | 399 ± 130 | 3.1 ± 0.4 |
| 7-OH-Warfarin | | |
| UGT1A1 | 58.8 ± 24 | 1.3 ± 0.1 |
| UGT1A10 | 220 ± 40 | 1.0 ± 0.1 |
| 8-OH-Warfarin | | |
| UGT1A8 | 206 ± 70 | 1.5 ± 0.1 |
| UGT1A9 | 107 ± 36 | 1.7 ± 0.1 |
| UGT1A10 | 383 ± 81 | 25 ± 2 |

### Determine the Inhibitory Potency of Each Enantiomer on Substrate Turnover.

As observed for R- and S-War for CYP2C918, the broad specificity of UGT enzymes may result in competitive inhibition between substrates and other OHWars, and thus decrease catalytic efficiency. Studies were proposed using purified stereoisomers to exclude possible enantiomeric interactions affecting the results as observed with racemic mixtures. It is possible that interaction between the R and S enantiomers could cause inhibition of glucuronidation kinetics observed with racemic OHWars. For example, stereoselective glucuronidation of S-OHWars by UGT1A10 may be the result of inhibition by R-OHWars. Glucuronidation activities for all expressed UGTs may be measured in vitro using HPLC techniques as previously described.

IC₅₀ studies may be carried out to investigate the inhibitory potential of enantiomeric OHWars toward UGTs involved in the metabolism of War. First, these UGTs may be assayed using S-OHWars (6-, 7-, and 8-OHWar) being inhibited by their respective enantiomer pair. These studies may be performed in vitro using UGT1A1, 1A3, -1A8, -1A9, and -1A10 recombinant enzymes. As a function of increasing inhibitor concentration, enzyme reaction velocities may be measured at a single substrate concentration equal to the respective Kₘ with increasing amounts of inhibitor to maximize signal response. Potent inhibitors may be identified based on these IC₅₀ values and may be further examined using Kᵢ studies, which may determine the affinity of inhibitory enantiomers for respective UGTs. These Kᵢ studies may vary the concentration of substrate while keeping the inhibitor constant. The steady-state kinetics of the potently inhibited UGTs may be assessed at multiple levels of inhibition by the enantiomers identified by IC₅₀ analysis.

Kinetic analysis (IC₅₀ and Kᵢ) of these experiments may determine the inhibitory potency of each enantiomer. These experiments may be analyzed and the R- and S-OHWar products may be separated on a chiral column using our existing HPLC method. All experiments may be confirmed using LC-MS/MS analysis.

### Separation and Purification of R and S War Enantiomers.

To accomplish this specific aim enantiomeric pure compounds are needed, but are not commercially available. UGTs, like CYPs, exhibit strong metabolic enantioselectivity with respect to the R and S enantiomers of War. The availability of pure enantiomers of OH-Wars may be critical for delineating the effects of OH-Wars on CYP and UGT activity and ultimately in the analysis of human metabolic profiles. These compounds may be needed as reagents in the proposed studies and as authentic standards in our LC-MS/MS method.

While the R and S enantiomers of War are available commercially, the OHWars are only available as racemic mixtures of the R and S enantiomers. To address this issue a preliminary semi-preparative HPLC based method was developed for the separation and purification of enantiomers of War and OHWars. The HPLC method utilizes the Chirobiotic™ V Chiral liquid chromatography column for the separation of enantiomers. FIG. 5 shows the separation of 2 mg of racemic 7-OHWar into 1 mg each of R-7-OHWar and S-7-OHWar. Enantiomers are collected separately, dried down, and resuspended in ethanol for catalytic studies. This HPLC based purification method may be adapted for the separation and purification of War and each of the OHWars to yield sufficient amounts of compounds for the proposed studies. Milligram quantities of pure enantiomers of each compound may be purified according to this method for use as reagents and as authentic standards.

Bio- and chemical synthesis of War and OHWar glucuronides. All hydroxylated glucuronides may be biologically synthesized for use as quantitive standards according to general methods described previously (19, 20). Semi-preparative scale biosyntheses may be carried out to produce the large amounts of enantiomerically pure OHWar-Glucs necessary for use as MS standards. Using cell lysates from baculovirus-infected insect cells expressing recombinant human UGT1A10 it was possible to produce milligram quantities of 8OHWar-Gluc. For the purpose of purification, reactions may be quenched by adding a 3-5 fold excess of glycine-trichloroacetic acid buffer, pH 2.8, and applied to C18 cartridges as previously described. Finally, a methanol solution containing unreacted substrate and glucuronide may be separated by preparative HPLC. Applying this protocol it was possible to achieve ˜95% yield for the biosynthesis of racemic 8-OHWar-Gluc. Chemical synthesis may be carried out via the Koenings-Knorr reaction. The deacetylation and crystallization of the final product may be carried out as described previously.

### Statistical Analysis

GraphPad Prism 4 (GraphPad Software, Inc., San Diego, Calif., USA) may be used for analysis of simple screening data. The results may be analyzed by one-way ANOVA followed by Dunnett's multiple comparison test. Measurements that vary significantly from the controls may be indicated. To analyze kinetic and inhibition reactions, the DynaFit program (BioKin, Ltd.) may be employed to fit and statistically compare data to model mechanisms incorporating one or two binding sites for substrates. For the single site mechanism, substrate turnover obeys the mechanism described by the traditional Michaelis-Menten kinetic scheme. For the two binding site mechanisms, one substrate binding site is active for glucuronidation, whereas the second site may or may not be competent for catalysis. Regardless of the catalytic potential of the secondary site, its occupation could impact catalysis allosterically as reported in the studies.

### Anticipated Results

Novel data may be generated on the glucuronidation of each individual R- and S-OHWar. It is anticipated that different kinetic parameters may be obtained for the separated enantiomers as compared to the racemic mixture. It is further anticipated that significant competition between individual enantiomers within the War binding site of UGTs may be observed, causing significant inhibition of the critical detoxification processes. Moreover, direct comparison of the glucuronidation processes of each enantiomer in hepatic and intestinal tissues may reveal the potential first pass sites of War detoxification. More importantly, these in vitro data may give insights into new drug targets. This information may shed light on the potential advantages of the use of enantiomerically pure War as the drug of choice in War therapy. This approach may eliminate at least 50% of the potential metabolites, therefore simplifying the biotransformation pathway and potentially eliminating adverse reactions caused by the racemic mixture. Lastly, these studies may generate previously unavailable compounds for quantitatively investigating the contributions of these enzymes to War inactivation and elimination, and thus impact proposed in vitro & in vivo studies.

In view of the fact that these studies may also provide methodology for the production of large amounts of pure enantiomeric compounds never before available for study, this technology could be expanded for use in the production of the enantiomerically pure drug.

### Example 3

An important contributor to patient metabolic capacity is the ability of molecules to compete with War for enzyme active sites. Concurrent drugs that recognize the same CYPs metabolizing War have been shown to greatly affect R- and S-War clearance and drug responses. Similarly, it is hypothesized that OHWars act through a product feedback inhibition mechanism on CYP hydroxylation of War, thereby decreasing catalytic efficiency. OHWar binding or re-binding to metabolizing enzymes effectively blocks subsequent turnover of the parent drug, thereby decreasing clearance and enhancing the anticoagulant effect (FIG. 6A). The significance of this mechanism derives from patient metabolic capacities for CYP activities, such that higher capacities favor inhibition, while lower capacities do not. The potency of this effect is underscored by higher bioavailability of OHWars relative to War due to weaker plasma binding properties. As a counter to product accumulation, OHWars undergo Phase II conjugation by UGTs and SULTs leading to formation of glucuronide and sulfated conjugates readily recognized by transporters for urinary excretion. Evidence for Phase II metabolism has been shown through studies with rat hepatocytes, recombinant human UGTs17, and patient urine samples. As illustrated in FIG. 6B, it is further hypothesized that the action of Phase II enzymes on OHWars suppresses product inhibition of CYPs during War metabolism.

Preliminary IC₅₀ studies for CYP1A2, CYP2C9*1, and CYP3A4 with P450-Glo marker reactions demonstrated the inhibitory potency of OHWars toward the parent enzyme as well as other CYPs (Table 6). CYP1A2 products 6- and 7-OHWar retained effective binding to the enzyme following their production. Surprisingly, CYP3A4 generated 4′-OHWar, which displayed higher affinity (lower IC50) than substrate War for CYP3A4. Interactions between CYP reactions were suggested by potent inhibition of CYP2C9*1 by CYP3A4 products, 10- and 4′-OHWar. Moreover, 8-OHWar from CYP1A2 activity competed effectively with War for CYP3A4.

TABLE 6
OHWar IC50 values (μM) for CYP marker reactions.

| | | 6-OH | 7-OH | 8-OH | 10-OH | 4′-OH |
|---|---|---|---|---|---|---|
| CYPs | War | War | War | War | War | War |
| CYP1A2 | 740 | 1400 | 400 | 830 | — | >3000 |
| CYP2C9*1 | 7.6 | 44.3 | 45.7 | 17.2 | 2 | 8.4 |
| CYP3A4 | 200 | >500 | >500 | 240 | — | 39 |

The impact of the generation and further processing of all five OHWars on CYP1A2, CYP2C9*1, CYP3A4 and CYP2C19 activities may be studied. CYP2C9*1 is the major metabolizer of S-War, while the other two specifically metabolize R-War. All three enzymes produce marker products, which make them ideal for monitoring alterations of metabolic profiles as a consequence of enzymatic shunting the impact of OHWar on this process may be determined by measuring IC₅₀ and Kᵢ values for recombinant enzymes and S9 fractions from human intestine and liver. The elimination of OHWars down specific Phase II pathways may be assayed by activating UGTs and/or SULTs with these same tissue fractions. Taken together, these studies would mark the first attempt to assess the collective impact of multiple CYP and Phase II processes on the pathways leading to the generation of War metabolic profiles.

### Experimental Approach

### Determine the OHWar Inhibitory Potency on Recombinant CYP Metabolism of War.

Potent OHWar interactions may be identified by carrying out IC₅₀ studies for recombinant enzymes against enantiomeric 6-, 7-, 8-, 10-, and 4′-OHWar. The effect of the inhibitors on S-War for CYP2C9*1 and R-War for CYP1A2 and CYP2C19 may be measured. Upon addition of NADPH to support CYP activity, initial velocities for substrate turnover may be measured at a substrate concentration equal to the Kₘ as a function of increasing OHWar inhibitor concentration using published reaction conditions. Product quantitation during the reaction may be accomplished through the LC-MS/MS method and the resulting data analyzed to determine the IC₅₀ value.

Based on the relative values of IC₅₀ to Kₘ, potent inhibitors may be further studied to determine OHWar inhibition constants (Kᵢ) toward preferred substrates using the same LC-MS/MS method the inhibition of steady-state substrate turnover may be measured at three inhibitor concentrations and then fit the data globally to determine the inhibition mechanism as well as the kinetic parameters (Vₘₐₓ and Kₘ) and Kᵢ for the reaction.

### Assess OHWar Inhibition of CYP Contributions to Metabolic Profiles Generated by Liver and Intestinal Fractions

While recombinant systems provide a simpler system for study, cellular fractions reflect the complex mix of enzymes that determine the flux of drugs to metabolites. Contributions of CYP1A2, CYP2C9*1, and CYP2C19 alone and in combination to War metabolic profiles generated by commercially available liver and intestinal S9 fractions containing Phase I CYPs and Phase II UGTs and SULTs may be measured. These fractions may be incubated with R- and S-War to determine steady-state parameters (Vₘₐₓ and Kₘ) for all OHWars present in the metabolic profile as described previously. The feedback inhibition mechanism may then be tested by repeating those steady-state experiments at different concentrations of enantiomeric 6-, 7-, 8-, 10-, or 4′-OHWar to yield Kᵢ values for each metabolic pathway. Due to the production of specific products by these CYPs, the Kᵢ values for S-7-OHWar, R-6-OHWar, and R-8-War may reflect the impact of the particular OHWars on reactions catalyzed by CYP2C9*118, CYP1A231, and CYP2C1932, respectively. These K values may be compared to those obtained by using recombinant enzymes.

### Alleviate OHWar Inhibition of CYP Activity Due to Action by Phase II Enzymes Present in Liver and Intestinal Fractions.

The effectiveness of Phase II enzymes to suppress product inhibition may be measured by utilizing the same liver and intestinal S9 fractions for coupled assays. The previous steady-state experiments may be repeated in the presence of an activated UGT and/or SULT system. Specifically, the Phase II system may be primed with the necessary cofactors, UDP-glucuronic acid for UGTs and 3′phosphoadenosine 5′-phosphosulfate for SULTs as described, before initiating the metabolism of S- or R-War by the CYPs. These cofactors may be included alone and in combination to assess the effect of each Phase II pathway and their combination on CYP-generation of OHWars. Due to the availability of glucuronide standards War, OHWars, and the corresponding glucuronides may be directly quantified by the LC-MS/MS method. By contrast, sulfate metabolite concentrations may have to be estimated indirectly by measuring the difference in levels of War and OHWars before and after treatment of samples with sulfatase. To determine if any bis conjugates form when UGTs and SULTs are active, samples may be treated with glucuronidase and sulfatase alone and in combination. If the summation of OHWars from individual digests is less than the amount of OHWars from the double digestion, then there are bis conjugates, which have been reported for rats. It is possible that CYP capacities could be too low to generate sufficient levels of OHWars to inhibit hydroxylation within the time frame of the experiment. Consequently, reactions may be spiked with R- or S-OHWar and measure the effect on War metabolism.

### Statistical Analysis

The analysis of the data requires common tools for fitting steady-state data using GraphPad Prism 4 (GraphPad Software, Inc., San Diego, Calif., USA). For the steady-state studies, velocities may be determined by linear regression. The resulting plot of these velocities as a function of substrate concentration may be fit to the Michaelis-Menten equation. To assess the mechanism of inhibition, DynaFit software (BioKin, Ltd.) may be used to fit and statistically compare data to different reversible inhibition mechanisms (competitive, uncompetitive, and mixed). The most optimal model may be used for determining kinetic parameters for the OHWar inhibition of the respective reaction.

### Anticipated Results

These studies may provide the first insight toward the importance of hydroxylation and conjugation capacities for War metabolism. These efforts may identify the enantioselectivity of OHWar inhibitors for CYP1A2, CYP2C9*1, and CYP2C19. Preliminary results from IC₅₀ studies already indicate the significance of the site of hydroxylation on affinity toward these enzymes. The association of OHWars with these CYPs may alter enzymatic capacities and consequently specific metabolites of reaction profiles generated by intestinal and liver fractions. In addition to interactions between CYP activities, the inclusion of UGT and SULT processing of OHWars may mark the first attempt to explore the role of coupling between Phase I and II processes that contribute to War clearance and presumably metabolic profiles in patient urine. Follow up studies could focus on identifying specific UGTs and SULTs responsible for conjugating OHWars and through this process construct metabolic pathways for War. Nevertheless, the use of steady-state conditions may obscure the impact of feedback inhibition due to the lack of product accumulation. Alternatively, progress curves could be substituted for these analyses by monitoring reaction progression at a set substrate concentration until the end point is reached. The effectiveness of Phase II elimination of OHWar inhibition may result in higher levels of substrate conversion as indicated by the endpoint of the reaction. In the context of drug-drug interactions, the validation of this strategy may be invaluable for future studies assessing the role of competing drugs in shunting War metabolism down alternate metabolic pathways indicated by altered metabolic profiles. Lastly, it may be possible to construct kinetic models to understand the relationships between these enzymes and the resulting metabolic profiles, which may prove invaluable for analyzing profiles from patients undergoing War therapy. This information may be vital to development more robust anticoagulant treatment strategies.

### Example 4

Unlike genotyping and other approaches, metabolomics reveals the metabolic phenotype of patients and thus accurately reflects the true capacity of patients to transform War into inactive metabolites. Nevertheless, the potential for this approach remains unrealized. Early efforts to identify suitable biomarkers for intra- and inter-patient variability were not successful due to the emphasis on free OHWar levels, which ignores the context of CYP activities in War metabolism. The availability of OHWars reflects their generation by CYPs and disappearance through Phase II conjugation. While the role of CYPs in War metabolism has been studied extensively, little is known about Phase II metabolism. Similar to CYPs, recombinant UGTs demonstrate regioselective and possibly enantioselective metabolism of OHWars. Variations in these activities among patients would alter OHWar levels and consequently compromise attempts to identify relevant biomarkers for War treatment. Total OHWar levels are required for pursuing metabolomic strategies, and the inclusion of Phase II metabolites is clearly warranted. The respective levels of these metabolites reflect the interplay between these processes and corresponding enzymes whose contributions depend on the capacity to catalyze the particular metabolic step. Genetic polymorphisms of metabolizing enzymes, e.g. CYP2C9, affect metabolic capacity as well as clinical factors. The collective contributions of these effects are captured in the levels of the metabolites.

Due to the potentially large size of the War metabolome (>36 metabolites), a specific, sensitive method is necessary for quantitative studies. The solution is an LC-MS/MS approach, which quantitatively measures enantiomers of War, OHWars, and corresponding glucuronides and sulfates in human plasma and urine.

Plasma is a commonly used matrix for studying drug metabolism including War; however, the ultimate goal is to develop a diagnostic approach tailored to urine samples from patients due to the noninvasiveness, high volume, and minimal infectious disease risk to patients and clinicians. Consequently, both types of samples may be obtained and their suitability compare as a source of biomarkers for the impact of clinical factors on War metabolism. By focusing on the end products of metabolism, the personalized metabolic phenotype for each patient is revealed as an accurate reflection of the environmental and genetic factors affecting War metabolism and consequently patient drug response.

Preliminary analysis of urine metabolic profiles for three War-treated patients identified 22 unique metabolites and revealed the diversity of metabolic profiles, i.e. OHWars (red) and Glucs (green), and corresponding levels among patients (FIG. 7). Based on total OHWar levels, S-70HWar was the predominant metabolite indicating the significance of CYP2C9 in S-War metabolism. R-6- and 8-OHWar levels demonstrated R-War metabolism was mainly due to CYP1A2 and CYP2C19, respectively, but contributions differed among patients. Subsequent processing to glucuronides was highly dependent on the respective patient and supported the feedback inhibition mechanism (FIGS. 6A&B). Patient F84 excreted mostly glucuronides (59%) reflective of a high Phase II capacity that likely increased CYP activities and correspondingly generated high levels of metabolites. By contrast, the lower UGT capacity observed for patient F87 resulted in ˜three-fold lower total metabolites. Unlike 4″- and 8-OHWar, 6- and 7-OHWar were primarily (>70%) excreted as glucuronides. UGT1A1 was likely responsible for 6-OHWar-Gluc production, although UGT1A10 possibly contributed for patient F87. Five different UGT1A enzymes potentially generated the 7-OHWar-Gluc levels seen. More studies clearly are needed to validate the role of these metabolites as biomarkers and consequently drug response related to metabolism.

As a step toward this goal, the following may be performed 1) the method to improve throughput and assess the matrix effect (plasma versus urine) may be further developed, 2) contributions of clinical factors to metabolic profiles may be validated, 3) metabolites correlating with dosage may be identified, 4) the role of Phase II metabolism on patient CYP metabolic capacities may be confirmed, and 5) metabolic profiles into enzymatic signatures may be translated to identify specific contributions of enzymes. After improving the analytical method, the metabolic profiles from matched plasma and urine samples may be determined for 300 War patients obtained commercially. These samples may represent populations with sufficient genetic diversity and contributions of common clinical factors (gender, age, ethnicity, and concurrent medications) to enable correlative studies. In addition, the hypothesized role of Phase II metabolism in suppressing CYP product inhibition and thus increaseing CYP generation of OHWars may be tested. Specifically, the direct correlation between Phase II capacities to generate metabolites and overall CYP contributions to War metabolism may be validated. All correlative studies may benefit from the generation of specific products by CYP1A2, CYP2C9*1, and CYP2C19 and possibly others. Relationships between metabolites may then be assigned to specific enzymes that ultimately contribute to the complex metabolic pathways of War.

It is believed that the knowledge generated by analyzing and quantifying those metabolites may provide critical guidance during War treatment to 1) minimize the time necessary to optimize dose, 2) decrease treatment costs, and 3) potentially avoid harmful War-drug interactions.

### Experimental Design

### Further Develop LC-MS/MS Analytical Method for Determining Patient Metabolic Profiles

Metabolic profiles may be generated for 300 matching plasma and urine samples (ProMedDx, Inc) from War treated patients by the original LC-MS/MS method or an improved version. All samples may be collected from patients who provided informed consent with strict IRB and HIPAA compliance. Rather than the current two injection approach, a single injection of these samples would decrease time and save money, and consequently alternative methods may be explored for determining metabolic profiles. A two dimensional HPLC with both columns set up in tandem may be explored to separate all compounds from one injection employing several published strategies. Alternatively, enantiomerically pure standards may be used and only the ZORBAX Eclipse XDB-C18 column employed to separate racemic forms of the metabolites. The levels of respective enantiomers may be quantitated using an on-line circular dichroism (CD) detector (Jasco, Inc) as demonstrated by others.

For the original method, an Agilent 1100 HPLC system coupled to an API4000 Triple Quadrupole mass spectrometer (Applied Biosystems) may be used to analyze and quantify WAR, OH-Wars, and the corresponding glucuronide and sulfated conjugates for patient samples reverse-phase HPLC using a ZORBAX Eclipse XDB-C18 column (Agilent) may be used to resolve War and OHWars without regard to chirality. Coupled with the chiral analyses, knowledge of the total amount of racemic metabolites enables the calculation of R and S forms of all metabolites, which was confirmed by standard MS/MS tools. Previously, glucuronides were estimated by treating samples with β-glucuronidase and measuring the increase in levels of their precursors, War and OHWar. This strategy may be used to determine levels of sulfated metabolites for the first time. Representative chromatograms of metabolites are shown in FIG. 8. It is anticipated that obtaining levels of respective metabolites for each patient to be further subjected to statistical analyses in order to reveal the patients' metabolic capacities toward War.

For subsequent correlative studies, the levels of urine metabolites may be normalized to adjust for variations in hydration of patients and time since last urination. Specifically, the metabolites may be referenced to creatinine as measured by a commercially available kit (Oxford Biochemical Research) or specific gravity using a standard refractometer. Although both are comparable methods, specific gravity may be useful if creatinine values are highly variable or low (37). Plasma samples may not require this type of normalization.

### Validate Contributions of Clinical Factors on Metabolic Profiles.

The resulting metabolite levels for each patient profile may be subjected to pair-wise comparison to common clinical factors: gender, age, ethnicity, and concurrent drugs. The projected range for the age of patients may be from 40 to 90 years old, which is the common range for donors to commercial specimen banks. Patients may be divided by ethnicity into Caucasians, African-Americans, and Asians for these studies. Lastly, patient samples may be further divided into populations with respect to medications known to potentiate War, e.g. low dose aspirin, amiodarone, histamine blockers, etc.

Identify Metabolites Correlating with Dosage.

War dosage is a product of the need to balance metabolic capacity with maintaining a steady-state War concentration. Consequently, a similar pair-wise analysis may be used to identify which metabolites are significant determinants of War inactivation and clearance based on correlations with dosage.

### Confirm the Role of Phase II Metabolism on Patient CYP Metabolic Capacities.

To validate the feedback inhibition mechanism in patients, pair-wise correlations may be formed between levels of OHWars and Phase II metabolites. These studies may be performed for metabolites within the same metabolic pathways, e.g. S-7-OHWar and S-7-O-Gluc, as well as those that are not to explore interactions between different pathways, as suggested by the inhibition studies (Table 6).

### Translate Metabolic Profiles Into Enzymatic Signatures to Identify Specific Contributions of Enzymes.

As a complement to studies for metabolites, the specificity of certain enzymes was used to generate marker products and assign relationships identified for metabolites to the parent enzyme. In effect, metabolic profiles may be translated into enzymatic profiles reflective of the personalized metabolic capacities for each patient. The significance of S-7-OHWar reflects CYP2C9 contributions, while R-6- and 8-OHWar mark CYP1A231 and CYP2C1932 activities, respectively. Through this approach, the metabolic pathways and their interactions to the overall clearance of War may begin to be constructed.

### Statistical Analysis

This is a retrospective, investigative pilot study and is exploratory in nature. Descriptive summaries may be provided for patient demographic and clinical characteristics. For continuous variables, the summaries may include sample size, mean and standard deviation (or median and interquartile range), and minimum and maximum. For categorical variables, the summaries may include frequencies and percentages. The coefficient of variation (i.e., Standard Deviation/Mean×100) may be calculated for each presented metabolite. Pair-wise correlations may be computed for each set of metabolites within each phase of metabolic steps; pair-wise correlations may also be computed for each individual metabolite within each Phase I against each individual metabolite within Phase II. Furthermore based on the findings from the pair-wise comparison analyses, partial correlations may additionally be computed between individual Phase I metabolites and selected subsets of Phase II metabolites. Between-group comparisons within each clinical factor (based on metabolite levels within each individual phase) may be made using t-tests and ANOVAs. Both simple (using individual clinical factors) and multiple regression models (including but not limited to a multiple stepwise regression analysis) may be conducted to determine the relative effects of age, gender, ethnicity, and concurrent medications on the War clearance based on each metabolic level. If a variable is not normally distributed in any of the analyses listed above and an appropriate transformation cannot be obtained, a non-parametric test may be used for the analysis. Analyses may be conducted in SAS and all p-values may be reported.

### Sample Size Justification

This is a retrospective, investigative pilot study and is exploratory in nature. The findings of this study may be based on 300 purchased serum and urine samples. Considering the proposed multiple regression model, a sample size of 300 may achieve 99% power to be able to detect an R-square value of at least 0.20 attributed to 4 independent variables using an F-test with an alpha level of .00016 (adjusted for multiple tests: 0.05/30=0.0016). It is very likely that the proposed sample size of 300 may be sufficient to provide an evident exploration of the stated study aims.

### Anticipated Results

The potential of the metabolomic approach to reveal the metabolic capacities of each patient and consequently provide a foundation for designing a dosing algorithm that avoids complications related to individual variations in the efficiency of patients to inactivate and eliminate War may be demonstrated. The transition to a single injection method may improve throughput, an essentially quality for a clinical tool. In addition, the demonstration of the feasibility of urine sampling over plasma may greatly facilitate testing of patients. The benefit of metabolic phenotyping of those patients may be apparent by the demonstrating of the effects of clinical factors on metabolite levels. Insight may be gained on the effects of age on War clearance, which remains a controversial topic. An early report suggested that metabolism was unaffected by age; rather, aging patients developed a sensitivity to the drug and thus require lower doses. By contrast, recent studies explain this observation through diminishing metabolic capacity as a function of age. Ethnicity clearly impacts War dosage. Because War sensitivity is linked, in some cases, to genetic variations in CYP2C9, It is expected to replicate the percent representation of this population within each ethnic group. Nevertheless, an even larger subpopulation may be identify based on the incorporation of more potentially polymorphic genes encoding metabolizing enzymes as well as other ethnic-dependent factors unrelated to genetic composition. There are a numerous of studies exploring potential drug-drug interactions related to concurrent medications prescribed with War. The sample set in this study may include many patients on multiple drugs, which poses an excellent opportunity to explore these interactions as well as a challenge due to the typical presence of more than one concurrent medication. In addition to clinical factors, the first investigation of the impact of sequential processing of War by Phase I CYP and Phase II enzymes on its clearance from patients may be provided. Due to the preliminary evidence on feedback inhibition, it is anticipated that a direct correlation of Phase II metabolic capacities of patients to translate into higher overall levels of total OHWars. As the primary recognized inactivation step, the increase in War hydroxylation by CYPs would impact dose response, a prediction that may be explored in future studies. Taken together, the efforts discussed in this proposal may provide a critical foundation to develop an algorithm for utilizing metabolic information as guidance for War doses that balance the metabolic potential of the patient with the appropriate anticoagulant effect.

### Example 5

### Contributions of Three CYP3A Family Members to R- and S-Warfarin

Coumadin (R/S-warfarin) is highly efficacious in the prevention of thromboembolic events associated with atrial fibrillation and stroke, but is widely underutilized due to fear of hemorrhage. Individual dosing is confounded by multiple factors including age, genetic polymorphisms, nutritional status, concomitant health status, drug-drug interactions etc. Most of these factors reflect contributions from drug metabolism. Multiple cytochrome P450s (CYP) hydroxylate warfarin into less active or inactive metabolites. These metabolic activities then lower levels of the active drug, and therefore impact drug response and reduce toxicity. Consequently, an understanding of warfarin metabolism is an important goal for improving patient outcomes during therapy.

CYP3A4 is the most abundant hepatic isoform, and is known to contribute to R- and S-warfarin metabolism, forming both 10-hydroxywarfarin and 4″-hydroxywarfarin metabolites. The biological significance of CYP3A4 activity toward warfarin remains controversial. Warfarin-drug interactions purportedly to depend solely on CYP3A4 activity have yielded conflicting reports. Moreover, expression of CYP3A4 is highly variable between individuals, with up to 100-fold differences in expression and activity. Most studies examining the generation of the 10- and 4″-hydroxywarfarin metabolites in plasma derived metabolic profiles ascribe such activity to CYP3A4, but the specific role of CYP3A4 in drug metabolism may be complicated by the presence of two other CYP3A subfamily members, CYP3A5 and CYP3A7.

CYP3A5 and CYP3A7 are known to share closely overlapping substrate specificity with CYP3A4 toward multiple xenobiotic compounds due to high sequence homology among the enzymes (up to ˜90%). A comparison of the metabolic capacities of these three isoforms with respect to ten common drug substrates revealed important differences in affinity and metabolic efficiencies toward those substrates. A similar relationship may exist for all three enzymes toward warfarin metabolism such that activities attributed to CYP3A4 actually reflect the combined effect of the CYP3A family. Although little is known about CYP3A7, polymorphisms of CYP3A5 have recently been associated with warfarin sensitivity in genetic studies. Consequently, it is hypothesized that CYP3A5 and CYP3A7 contribute to R- and S-warfarin metabolism and thus modulate the role of CYP3A in the CYP mediated metabolism of warfarin. It is anticipated that the combined effects of these activities contribute to the clinical response to warfarin therapy.

In this study, the hypothesis was tested by determining the in vitro steady-state kinetics for R- and S-warfarin metabolism by recombinant CYP3A5 and CYP3A7. For comparative purposes, recombinant CYP3A4 was included in these studies. Given the lack of specific inhibitors or antibodies for these enzymes, the studies were not broadened to more complex microsomal fractions. Taken together, these studies provided valuable insights regarding the potential impact of CYP3A5 and CYP3A7 on known and unexplored biological roles for CYP3A activity on warfarin response during therapy.

### Materials and Methods

### Materials

All chemicals used in this study were of at least reagent grade. Unless otherwise specified, all chemicals and reagents were purchased from either Sigma-Aldrich (St. Louis, Mo.). In particular, substrate (R-warfarin and S-warfarin), internal standard (7-hydroxycoumarin), and all hydroxywarfarin standards used for identification and quantification (6-hydroxywarfarin, 7-hydroxywarfarin, 8-hydroxywarfarin, 10-hydroxywarfarin, and 4′-hydroxywarfarin) were purchased from Sigma-Aldrich. Human recombinant CYP3A4, CYP3A5, and CYP3A7, expressed in baculovirus-infected insect cells (supersomes), were purchased from BD Biosciences (San Jose, Calif.).

### Steady-State Metabolism of R- and 5-Warfarin by CYP3A Enzymes

Each recombinant enzyme CYP3A4, CYP3A5, CYP3A7, was incubated at a concentration of 50 nM with either R- or 5-warfarin ranging from 2 to 250 μM in 50 mM potassium phosphate pH 7.4, at 37° C. Reactions were initiated upon addition of an NADPH regenerating system [2 μU μL-1 glucose 6-phosphate dehydrogenase, 10 mM glucose 6-phosphate, 2 mM MgCl₂, 1 mM NADP⁺]. Reactions were quenched with an equal volume of 0.4 M perchloric acid containing the internal standard (7-hydroxycoumarin, 20 μM) at 10 min for R-warfarin or 20 min for 5-warfarin respectively. The quenched reactions were centrifuged and the resulting supernatant containing substrate and products resolved and quantified by UPLC-MS/MS. Each experiment was performed in at least 4 independent experimental replicates.

### UPLC-MS/MS Analysis of Kinetic Reactions

An Acquity UPLC® (Waters, Milford, Mass.) system coupled to a TSQ-Quantum Ultra triple-quadrupole mass analyzer (ThermoFinnigan, San Jose, Calif.) was used to assess warfarin metabolite formation. All five potential warfarin metabolites, i.e. 6-, 7-, 8-, 10-, and 4″-hydroxywarfarin, were resolved by UPLC-MS/MS using a 2.1 mm×150 mm Acquity BEH C18 1.7 μm particle column (Waters) operating at 40° C. (FIG. 9).

A gradient method using H₂O (0.01% formic acid v/v) (A) and methanol (B) eluted bound metabolites as follows: 50% B from 0 to 0.5 min, with a linear gradient to 99% B from 0.5 to 3.5 min, returning to 50% B from 3.5 to 4.0 min, and held at 50% B for 5 min. The flow rate was constant at 250 μL/min. All metabolites and standards were detected in selected reaction monitoring mode (SRM) using the following m/z transitions: 325.1/257.1 (4′-hydroxywarfarin), 325.1/251.1 (10-hydroxywarfarin), 325.1/179.1 (6-, 7-, and 8-hydroxywarfarin). Electrospray conditions were as follows: spray voltage of 4500 V, sheath gas pressure 45, auxiliary gas pressure 20, and collision energy of 15-15 eV (compound specific).

### Determination of Kinetic Parameters

Final metabolite concentrations were calculated from the ratio of metabolite area to internal standard area in the sample and compared to calibration curves as determined from the UPLC-MS/MS method. All compounds were quantified relative to authentic standards. The resulting kinetic data were analyzed with GraphPad Prism 5® software. Non-linear regression analysis was used to fit steady-state data to the Michaelis-Menten equation to determine the Vmax and Km parameters. 95% Confidence intervals for both parameters were also determined as shown in Table 7 and Table 8.

TABLE 7
Kinetic parameters of R-warfarin metabolism by CYP3A enzymes
Metabolism of R-Warfarin

| | 10-Hydroxywarfarin | | 4′-Hydroxywarfarin | | | | |
|---|---|---|---|---|---|---|---|
| | Vₘₐₓ* | Kₘ (μM) | Vₘₐₓ/Kₘ | Vₘₐₓ | Kₘ (μM) | Vₘₐₓ/Kₘ | 10OH:4′OH^{&} |
| CYP3A4 | 1055 | 16 | 66 | 98 | 36 | 2.7 | 24:1 |
| | (971-1139) | (11-20) | | (85-111) | (21-50) | | |
| CYP3A5 | 263 | 129 | 2.04 | 108 | 244 | 0.44 | 4.6:1 |
| | (159-366) | (24-233) | | (0-221) | (0-657) | | |
| CYP3A7 | 116 | 54 | 2.15 | 260 | 1389 | 0.19 | 11:1 |
| | (91-141) | (23-85) | | (0-1069) | (0-6348) | | |

*Units for Vₘₐₓ in pmol product/min/nmol P450
^{&}This represents the ratio of10-hydroxywarfarin efficiency (Vₘₐₓ/Kₘ) to 4′hydroxywarfarin efficiency (Vₘₐₓ/Kₘ)
( ) indicated 95% confidence intervals

TABLE 8
Kinetic parameters of S-warfarin metabolism by CYP3A enzymes
Metabolism of S-Warfarin

| | 10-Hydroxywarfarin | | 4′-Hydroxywarfarin | | | | |
|---|---|---|---|---|---|---|---|
| | Vₘₐₓ* | Kₘ (μM) | Vₘₐₓ/Kₘ | Vₘₐₓ | Kₘ (μM) | Vₘₐₓ/Kₘ | 10OH:4′OH^{&} |
| CYP3A4 | 76 | 26 | 2.9 | 64 | 27 | 2.4 | 1.2:1 |
| | (65-86) | (15-37) | | (53-75) | (12-42) | | |
| CYP3A5 | 67 | 318 | 0.21 | 157 | 672 | 0.23 | 0.9:1 |
| | (0-181) | (0-1165) | | (3.2-311) | (0-1521) | | |
| CYP3A7 | 22 | 81 | 0.27 | 321 | 2283 | | |
| | (18-26) | (45-118) | | (0-805) | (0-6038) | 0.14 | 1.9:1 |

*Units for Vₘₐₓ in pmol product/min/nmol P450
^{&}This represents the ratio of 10-hydroxywarfarin efficiency (Vₘₐₓ/Kₘ) to 4′hydroxywarfarin efficiency (Vₘₐₓ/Kₘ)
( ) indicated 95% confidence intervals

### Results

Product linearity with respect to time was confirmed for all enzymes and products for the time points used in this study. Each of the three recombinant enzymes (CYP3A4, CYP3A5, and CYP3A7) generated 10-hydroxywarfarin and 4′-hydroxywarfarin from both R- and S-warfarin, but with significantly different kinetic parameters (FIGS. 10 and 11). Given the capability of the method to detect a reaction velocity of ˜0.2 pmol product/min/nmol P450 and that no other hydroxywarfarin products were observed, less than 0.9% of products could have been other hydroxywarfarins even in the slowest reactions.

For R-warfarin, each isoform generated R-10-hydroxywarfarin more efficiently than R-4′-hydroxywarfarin with significant differences among the enzymes with respect to the corresponding Vₘₐₓ and Kₘ parameters. CYP3A4 displayed the highest Vₘₐₓ, approximately four and nine times higher than CYP3A5 and CYP3A7 respectively (Table 7). CYP3A4 also displayed the highest affinity for R-warfarin, having a Kₘ approximately eight and three fold lower than CYP3A5 and CYP3A7 respectively. CYP3A5 and CYP3A7 yielded nearly equal metabolic efficiencies (Vₘ/Kₘ) with respect to R-10-hydroxywarfarin, but CYP3A4 was approximately 33-fold more efficient in the production of R-10-hydroxywarfarin. The secondary metabolite, R-4′-hydroxywarfarin, was produced with lower maximal rates, with the exception of CYP3A7 which generated R-4′-hydroxywarfarin with a Vₘₐₓ approximately 2.25-fold higher than for generation of R-10-hydroxywarfarin. This metabolite was also generated in a much lower affinity reaction for each of the three enzymes, with CYP3A4, CYP3A5, and CYP3A7 generating this secondary metabolite with approximately 24, 5, and 11-fold lower efficiency than for R-10-hydroxywarfarin respectively. When comparing the metabolic efficiencies for the respective metabolic pathways, all CYP3A isoforms favored 10-hydroxylation over 4′-hydroxylation such that CYP3A4 displayed the highest preference and CYP3A5 the weakest preference (Table 7, 10-OH:4″-OH).

Despite identical regiospecificity (oxidation at the same sites of warfarin), S-warfarin metabolism was less efficient than R-warfarin metabolism for all CYP3A enzymes. CYP3A4 demonstrated nearly equal maximal velocities for both hydroxywarfarin products while CYP3A5 generated 4″-hydroxywarfarin with a greater than two-fold higher rate (Table 8). CYP3A7 generated 4′-hydroxywarfarin with a higher maximal rate than 10-hydroxywarfarin, but demonstrated extremely poor affinity (Kₘ) toward S-warfarin substrate. Each of the three recombinant enzymes generally displayed lower affinity for S-warfarin than R-warfarin as demonstrated by the higher Kₘ values for each metabolite (with the exception of CYP3A4 generation of 4′-hydroxywarfarin). Together, these lower maximal activities and lower affinities, as compared to R-warfarin metabolism, result in lower metabolic efficiency toward S-warfarin. CYP3A isoforms were much less selective toward hydroxylation of S-warfarin at the 4′- and 10-hydroxyl positions as compared to R-warfarin hydroxylation.

### Discussion

The in vitro studies presented here provide the first analysis of the capacity of CYP3A5 and CYP3A7 to metabolize R- and S-warfarin as well as complement previous kinetic studies for CYP3A4. CYP3A4, CYP3A5 and CYP3A7 metabolized R- and S-warfarin to the same products, 10- and 4′-hydroxywarfarin. Given the high sequence homology and similar activities for certain drugs, the overlapping regio- and enantio-specificity among the three CYP3A enzymes is plausible. The unique metabolic efficiencies for the respective reactions indicated that each isoform impacts warfarin differently. These results suggest that 10-hydroxywarfarin is a marker of global CYP3A activity rather than just CYP3A4 as previous reports have suggested. 4′-Hydroxywarfarin is a non-specific marker of CYP3A activity because it is also generated by CYP2C8, CYP2C9, and CYP2C19. Overall, CYP3A5 and CYP3A7 displayed significantly decreased metabolic efficiency for 10-hydroxywarfarin generation from R-warfarin as compared to CYP3A4. This decreased efficiency toward R-warfarin is in agreement with the general trend that CYP3A5 and CYP3A7 are less efficient than CYP3A4 for most substrates .

The reduced efficiencies of CYP3A5 and CYP3A7 compared to CYP3A4 observed in this study suggests that individuals who have lower relative CYP3A4/CYP3A content might display increased sensitivity to warfarin or potentiation of CYP3A mediated drug-drug interactions. In addition to anticoagulant therapy, concurrent ailments, often require individuals to be prescribed other drugs, which are metabolized by CYP3A enzymes, and thus potentiate warfarin-drug interactions. Adverse interactions between warfarin and several drugs, including diltiazem, quinidine, cimetidine, and cisapride, have been observed in vivo that are not due to other effects, such as interactions between S-warfarin metabolism and CYP2C9, a major drug metabolizing enzyme. Many of these drugs are known to be metabolized by CYP3A4 and possibly by CYP3A5 and CYP3A7. Genetic variability and changes in protein expression with respect to CYP3A5 and CYP3A7 may contribute to the warfarin metabolism and response to therapy and thus explain conflicting reports on the importance CYP3A4 during anticoagulant therapy.

CYP3A5 expression is highly variable and may represent more than 50% of total CYP3A content in some individuals. The CYP3A5*1 allele confers high levels of expression and activity, while the common *3 and *6 alleles have no activity. A large prospective study examining the relationship of 29 different genetic factors on warfarin therapy in British patients found a significant association between CYP3A5 and sensitivity to warfarin, defined as a maintenance dose of ≦1.5 mg mg/day. These results suggest that CYP3A5 is an important factor in the metabolism of warfarin in this population. The decreased metabolic capacity of CYP3A5 toward warfarin observed in the current study provides a mechanistic explanation for the in vivo association of CYP3A5 with warfarin sensitivity. In contrast, an earlier pharmacogenomic study did not find a statistically significant association between CYP3A5 genotype and maintenance dose or bleeding complications in a Swedish population. However, this study utilized a small cohort (only 2 cases) of individuals with the active CYP3A5*1/*1 genotype. The majority of patients (88%) were homozygous for the inactive CYP3A5*3 allele, such that CYP3A5 did not contribute to CYP3A metabolism of warfarin, further bolstering the observation that CYP3A5 contribution to warfarin metabolism potentiates drug response. Together these data suggest that CYP3A5 contribution, partly determined by genotype, may be an important risk factor in warfarin therapeutic outcomes.

Recent investigations have identified high expression of CYP3A7 in approximately ten-percent of adult livers, accounting for up to 36% of total hepatic CYP3A content in such individuals. The high level of CYP3A7 expression in these adults is associated with, but not limited to, the CYP3A7*1C allele. Outside of this population, CYP3A7 expression is thought to diminish to very low levels within the first year of life, with concomitant increases in expression of CYP3A4 and CYP3A5. Although regarded as a fetal specific isoform, CYP3A7 expression was detected in 100% of adult livers tested in a study examining the molecular mechanisms of CYP3A7 expression. However, the regulation of CYP3A7 expression in adults is not completely understood and the impact of CYP3A7 on warfarin therapeutic outcomes has not yet been investigated. Based on the current data, CYP3A7 may be a risk factor for warfarin sensitivity similarly to CYP3A5, especially in individuals possessing the CYP3A7*1C allele. The biological impact of CYP3A7 on warfarin therapy is likely more complex, because many drug-metabolizing enzymes undergo ontogenic changes before and after birth. Moreover, children are known to require higher doses than adults for unknown reasons.

### Conclusion

Based on these efforts, CYP3A5 and CYP3A7 likely contribute to in vivo metabolism of R- and S-warfarin, generating 10-hydroxywarfarin as a biomarker for total CYP3A activity toward the drug. The results of this study suggest that individuals with higher relative contribution by CYP3A5 and/or CYP3A7 compared to CYP3A4 are at risk for supra-therapeutic dosing and adverse bleeding events due to less efficient metabolism of warfarin. Individuals with high CYP3A5 expression or the CYP3A7*1C allele may especially require lower doses. The presence of these isoforms could also potentiate warfarin-drug interactions ascribed to the subtle differences in specificities among the enzymes.

### Example 6

### Hydroxywarfarin Metabolites Potently Inhibit CYP2C9 Metabolism of S-Warfarin

Coumadin (R/S-warfarin) is a highly efficacious anticoagulant for the prevention of thromboembolic events associated with atrial fibrillation and stroke. However, warfarin is widely underutilized due to fear of hemorrhage (1). Clinical use of the drug requires frequent monitoring due to its narrow therapeutic range and adverse consequences from supratherapeutic and subtherapeutic dosing. Warfarin is subject to drug-drug interactions, drug diet interactions and interpersonal variability due to metabolism (2). Efficient warfarin metabolism eliminates the active drug and decreases anticoagulant activity, while inefficient drug metabolism leads to toxic warfarin levels. Consequently, an understanding of processes that affect metabolic activity may improve the ability to explain variations in drug response and potentially avoid adverse effects during therapy due to metabolism.

Cytochrome P450s (CYP for specific isoforms) catalyze the first major step in warfarin metabolism by generating an array of hydroxywarfarins (FIG. 12). Although multiple CYPs metabolize warfarin, each enzyme exhibits regioselectivity and enantiospecificity for their respective reactions (3). CYP2C9 efficiently metabolizes S-warfarin, the more active form of the drug (4), into S-6- and 7-hydroxywarfarin (5), such that S-7-hydroxywarfarin is the most abundant metabolite in humans (6, 7, 8). Through a minor pathway, CYP3A4 hydroxylates S-warfarin to S-4′-hydroxywarfarin (5). Unlike S-warfarin, no single CYP dominates R-warfarin metabolism. CYP1A2 and CYP2C19 both generate R-6- and 8-hydroxywarfarin, but with differing regioselectivity (9, 10). CYP3A4 also metabolizes R-warfarin to R-10-hydroxywarfarin (11, 5), which is possibly the second most abundant plasma metabolite in humans (8). Clearly, warfarin hydroxylation involves a complex array of pathways involving different enzymes; nevertheless, these studies have focused mainly on the individual CYP reactions and not the combined effects of all reactions.

Similar to reported warfarin-drug/nutrient interactions (12), it is hypothesized here that hydroxywarfarins from CYP reactions compete with warfarin for CYP active sites and thereby suppress drug metabolism. The recognition of hydroxywarfarins by CYPs is plausible given the access to multiple sites for oxidation and overlapping specificities for warfarin among the enzymes regardless of whether oxidation occurs or not. Although R-warfarin is not metabolized by CYP2C9, this enantiomer binds and inhibits the enzyme with a Ki of ˜8 μM (13, 14). Further, warfarin alcohols are known to competitively inhibit CYP2C9, yet are not metabolized (15). Hydroxywarfarins may similarly retain affinity for the CYP2C9 active site. Hydroxywarfarin plasma levels may play also an important role in driving these inhibitory interactions. Racemic hydroxywarfarins associate with plasma proteins with approximately four-fold less affinity than warfarin (16, 17) suggesting that higher levels of unbound and hence inhibitory hydroxywarfarins are possible. The biological impact of these inhibitory interactions may also depend on variations in patient excretion of hydroxywarfarins or further metabolism to secondary glucuronide metabolites (5, 18).

The above hypothesis was tested by assessing the inhibitory potential of 6-, 7-, 8-, 10-, and 4′-hydroxywarfarin on S-warfarin metabolism by CYP2C9. These studies were carried out with racemic hydroxywarfarins because patients are dosed with racemic warfarin and produce mixtures of hydroxywarfarins (6). The P450-Glo™ marker reaction was employed to screen for potential inhibitors based on IC₅₀ values. Surprisingly, all hydroxywarfarins inhibited CYP2C9 activity, and thus the inhibitory potential of all racemic hydroxywarfarins was further assessed through subsequent steady-state kinetic studies with recombinant CYP2C9 and human liver microsomes. Contributions of other CYPs to S-warfarin metabolism were avoided by initially determining the inhibition mechanism and parameters for recombinant CYP2C9. The effect of the hydroxywarfarins on S-warfarin metabolism was subsequently studied using pooled human liver microsomes. Control studies with sulfaphenazole, a CYP2C9-specific inhibitor (19), validated the predominance of that enzyme in S-warfarin metabolism. It was then possible to attribute the inhibitory effect of the hydroxywarfarins to CYP2C9 present in the human liver microsomes.

### Experimental Procedures

### Materials

All chemicals used in this study were of at least reagent grade. Unless otherwise specified, all chemicals and reagents were purchased from either Sigma-Aldrich (St. Louis, Mo.) or Thermo Fisher Scientific (Waltham, Mass.). In particular, substrate (5-warfarin), internal standard (7-hydroxycoumarin), and all inhibitors (6-hydroxywarfarin, 7-hydroxywarfarin, 8-hydroxywarfarin, 10-hydroxywarfarin, and 4′-hydroxywarfarin) were purchased from Sigma-Aldrich. Ethyl alcohol (100%) was purchased from AAPER (Shelbyville, Ky.). Pooled human liver microsomes, human recombinant CYP2C9*1, expressed in baculovirus-infected insect cells (supersomes), and sulfaphenazole were purchased from BD Biosciences (San Jose, Calif.).

### IC₅₀ Studies on Hydroxywarfarin Inhibition of Recombinant CYP2C9 Activity

Hydroxywarfarins were screened for inhibitory potential toward recombinant CYP2C9 using the Promega (Madison, Wis.) CY2C9 P450-Glo™ Assay using the manufacturer's protocol. These IC₅₀ studies were analyzed on a PerkinElmer Victor³V™ 1420 Multilabel Counter. Recombinant CYP2C9, 50 nM, was incubated with 50 μM Luciferin H, and six concentrations of each hydroxywarfarin from approximately 10 nM to 4 mM final concentration, as optimized for each compound. The appropriate volume of hydroxywarfarin stocks in 100% ethanol were added to each well prior to addition of the substrate and enzyme, and the ethanol was allowed to evaporate at room temperature to minimize solvent effects on CYP2C9 activity. A final DMSO concentration of 1% was present in the reactions due to the P450-Glo Luciferin H. The reaction was initiated upon addition of an NADPH regenerating solution (BD Biosciences) and quenched with a luciferase quench (Promega) at 30 min. The resulting luminescence was measured at 25 min after quenching, and the IC₅₀ value was calculated with GraphPad Prism 5 (GraphPad Software Inc.) after scaling the data to 100% activity.

### Steady-State Metabolism of 5-Warfarin

The inhibition of 5-warfarin metabolism by recombinant CYP2C9*1 and human liver microsomes was carried out in the presence of each of the five racemic hydroxywarfarins. For recombinant CYP2C9, 25 nM enzyme was incubated with 0.5 to 64 μM S-warfarin in 50 mM potassium phosphate pH 7.4, at 37° C. Instead of recombinant enzyme, the human liver microsomal reactions contained 2.0 mg/mL protein. In both cases, reactions were initiated upon addition of an NADPH-regenerating system [2 μU μL⁻¹ glucose 6-phosphate dehydrogenase, 10 mM glucose 6-phosphate, 2 mM MgCl₂, 1 mM NADP+] and quenched with an equal volume of ethanol containing the internal standard (7-hydroxycoumarin) at 45 min. In most cases, hydroxywarfarin concentrations were varied five-fold above and below the IC50 value observed in the initial screen to optimize analysis of inhibitor data. In some cases, additional inhibitor concentrations were used to explore potential secondary binding sites suggested by uncompetitive and mixed inhibition mechanisms. For each hydroxywarfarin, at least 4 independent experimental replicates were performed. The quenched reactions were centrifuged and the resulting supernatant containing substrate and products resolved and quantified by LC-MS/MS.

### LC-MS/MS Analysis of Kinetic Reactions

The levels of 6- and 7-hydroxywarfarin in the reactions were analyzed with an Agilent 1100 HPLC system coupled to an API4000 Triple Quadupole (MS/MS) mass spectrometer (Applied Biosystems) using a 4.6×150 mm Zorbax Eclipse 5 μm XDB-C18 column (Agilent) at 40° C. A gradient method effectively separated all hydroxywarfarins and warfarin at a flow rate of 1 mL/min. The mobile phase consisted of (A) H₂O (0.1% acetic acid) and (B) methanol (0.1% acetic acid) with 50% A and 50% B held for the first 30 seconds, followed by a linear gradient from 50% B to 90% B (0.5-5.5 min), 90% B to 100% B (5.5-5.6 min), 100% B maintained for 2.5 min, 100% B to 50% B (7.9-8.0 min) with 50% B maintained for 2 min. MS/MS experimental conditions was used as described previously (6).

### Identification of Inhibitory Mechanism and Corresponding Parameters

The most probable inhibition mechanism for recombinant CYP2C9 and human liver microsome reactions was identified using DynaFit ver. 3.28 (Biokin, Ltd) (20). Data from each set of hydroxywarfarin experiments were fit to traditional reversible inhibition mechanisms (competitive, uncompetitive, noncompetitive, or mixed). The respective kinetic parameters (Kᵢ and Kᵢ′) were set as variables, while the kinetic parameters (Vₘₐₓ and Kₘ) were constants based on values obtained from uninhibited reactions. The most probable mechanism was then identified using the Akaike Information Criterion (AICc) for model discrimination (21).

### Results

### IC50 Values for Racemic Hydroxywarfarins Support Inhibition of CYP2C9 Activity

Based on P450-Glo assays, the initial inhibition screen for CYP2C9 activity demonstrated varying capacities for hydroxywarfarins to block enzyme activity (Table 9). Recombinant CYP2C9 was inhibited by racemic warfarin with an IC50 value comparable to the reported Kₘ of 2.6 μM (22). IC₅₀ values for the hydroxywarfarins ranged from approximately three-fold below to ten-fold above that measured for warfarin. 10-Hydroxywarfarin had the lowest IC50 toward CYP2C9, while 6-hydroxywarfarin had the highest. Intermediate affinity inhibitors were 8-hydroxywarfarin and 4′-hydroxywarfarin. Racemic 7-hydroxywarfarin yielded an IC₅₀ approximately eight-fold higher than that observed for warfarin.

TABLE 9
IC₅₀ values for racemic hydroxywarfarins toward recombinant
CYP2C9 activity^{a}
CYP2C9 Inhibitors

| | WAR | 4′OHWAR | 10OHWAR | 6OHWAR | 7OHWAR | 8OHWAR |
|---|---|---|---|---|---|---|
| IC₅₀(μM) | 4.5 | 9.3 | 1.6 | 47 | 35 | 16 |
| | (3.2-5.8) | (6.7-12) | (1.3-1.8) | (28-65) | (27-42) | (9.2-23) |

^{a}OHWAR = hydroxywarfarin;
95% confidence interviews shown in parentheses

Inhibition Kinetics of S-Warfarin with Recombinant CYP2C9

Steady-state inhibition experiments with recombinant enzyme validated the inhibitory potential of hydroxywarfarins on CYP2C9-mediated S-warfarin metabolism the uninhibited steady-state parameters for CYP2C9 toward S-warfarin were initially determined. For S-6-hydroxywarfarin, the Kₘ and Vmax parameters were 3.3 μM (2.6 to 4.0, 95% confidence interval) and 46 pmol/min*nmol P450 (43 to 49, 95% confidence interval) respectively, while the parameters for S-7-hydroxywarfarin were 2.3 μM (1.9 to 2.7, 95% confidence interval) and 68 pmol/min*nmol P450 (65 to 71, 95% confidence interval) for Kₘ and Vₘₐₓ respectively. These parameters were then used for subsequent inhibition studies. For 6-, 8-, 10-, or 4′-hydroxywarfarin studies, the effects of inhibition on formation of the major product, 7-hydroxywarfarin as the marker of CYP2C9 activity, were analyzed. Due to interference between the product and inhibitor, 7-hydroxywarfarin inhibition was studied based on 6-hydroxywarfarin formation. The concentration of inhibitors did not change significantly over the course of the reaction indicating minimal, if any, secondary metabolism. The most probable inhibition mechanism for each hydroxywarfarin was identified and listed in Table 10 based on model discrimination analysis between reversible inhibition mechanisms using DynaFit software. Mechanisms with high Akaike weights, but open confidence intervals were excluded from being the most likely mechanism of inhibition if another mechanism had sufficient support and closed confidence intervals.

TABLE 10
Racemic hydroxywarfarin inhibition of recombinant CYP2C9
metabolism of S-warfarin^{a}

| Inhibitor | Mechanism | Kᵢ (μM) | Kᵢ′ (μM) |
|---|---|---|---|
| 4′OHWAR | Mixed | 17 | 133 |
| | | (10-33) | (75-4113) |
| 10OHWAR | Competitive | 0.94 | — |
| | | (0.74-1.2) | |
| 6OHWAR | None | >170 | — |
| 7OHWAR^{b} | Competitive | 10 | — |
| | | (7.5-14) | |
| 8OHWAR | Mixed | 21 | 103 |
| | | (11-54) | (59-271) |

^{a}OHWAR = hydroxywarfarin; 95% confidence interviews shown in parentheses
^{b}Results based on S-6-hydroxywarfarin formation

These inhibition studies with recombinant CYP2C9 generated results similar to those observed for the IC₅₀ studies. Although kinetic analysis was performed with DynaFit, the data are shown as Hanes-Woolf plots to visually demonstrate the mechanism of inhibition. The Hanes-Woolf plots of 7- and 10-hydroxywarfarin (FIG. 12) support the competitive mode of action as indicated by the parallel lines for inhibited and uninhibited reactions. 10-Hydroxywarfarin was the most potent inhibitor of CYP2C9 with a K approximately 2.5-fold lower than the Kₘ for S-warfarin (Table 10). The second most potent inhibitor was 7-hydroxywarfarin demonstrating a Kᵢ 4.5-fold higher than the marker Kₘ, while no inhibition was observed with 6-hydroxywarfarin up to 170 μM inhibitor as indicated by the overlapping lines. Mixed inhibition was observed with both 4′- and 8-hydroxywarfarin, indicating that these metabolites inhibited CYP2C9 primarily through an intermediate affinity interaction, but also through a second low affinity (Kᵢ′>100 μM) interaction, which causes an increase in the slope of the Hanes-Woolf line (FIG. 12 and Table 10).

Inhibition Kinetics of S-Warfarin with Human Liver Microsomes.

CYP2C9 was the dominant CYP present in human liver microsomes that metabolized S-warfarin as reported by others (5). The steady-state kinetic profile was hyperbolic indicating the presence of a single enzyme capable of hydroxylating S-warfarin. The Kₘ and Vₘₐₓ kinetic parameters for 7-hydroxywarfarin from these studies were 5.2 μM (4.3 to 6.1, 95% confidence interval) and 173 pmol/min*mg protein (165 to 182, 95% confidence interval) respectively, which are comparable to those reported in the literature (3, 5). For S-6-hydroxywarfarin formation, the Kₘ and Vₘₐₓ kinetic parameters were 7.5 μM (6.4 to 8.6, 95% confidence interval) and 90 pmol/min*mg protein (86 to 94, 95% confidence interval) respectively. In the presence of sulfaphenazole, activity toward S-warfarin was potently inhibited, demonstrating a competitive inhibition constant of 2.5 μM (1.3 to 5.3, 95% confidence interval). Sulfaphenazole is reported to be specific to CYP2C9 at concentrations up to twenty-fold higher than used in this study (23), and the kinetic parameters found here are in agreement with previous reported values (24). These results confirm that S-warfarin is metabolized by a single microsomal enzyme, CYP2C9 under these experimental conditions.

The results from the human liver microsomes were very similar to those observed for the recombinant CYP2C9 studies (Table 11). For each hydroxywarfarin, the mechanism of inhibition was competitive, as demonstrated by the parallel lines in the Hanes-Woolf plots (FIG. 12). The most potent inhibitor of microsomal metabolism of S-warfarin was 10-hydroxywarfarin, the second most abundant metabolite in plasma (8). The corresponding Kᵢ was 2.4-fold lower than the observed microsomal Kₘ for S-warfarin (5.2 μM). Both 4′- and 8-hydroxywarfarin (FIG. 11) yielded intermediate affinity Kᵢ values that were approximately three-fold above the Kₘ for S-warfarin. The Kᵢ for 7-hydroxywarfarin was 8.5 fold higher than the marker Kₘ, which is in close agreement with the IC₅₀ results. 6-Hydroxywarfarin was a very weak competitive inhibitor of human liver microsomal activity, with an estimated Kᵢ approximately 32-fold higher than the Km for S-7-hydroxywarfarin (Table 11). Unlike the studies with recombinant CYP2C9, the apparent mechanism of inhibition for each hydroxywarfarin was competitive

TABLE 11
Racemic hydroxywarfarin inhibition of pooled human liver
microsome metabolism of S-warfarin^{a}

| Inhibitor | Mechanism | Kᵢ (μM) | Kᵢ′ (μM) |
|---|---|---|---|
| 4′OHWAR | Competitive | 17 | — |
| | | (13-24) | |
| 10OHWAR | Competitive | 2.2 | — |
| | | (1.7-3.0) | |
| 6OHWAR | Competitive | 167 | — |
| | | (117-257) | |
| 7OHWAR^{b} | Competitive | 64 | — |
| | | (55-74) | |
| 8OHWAR | Competitive | 13 | — |
| | | (11-16) | |

^{a}OHWAR = hydroxywarfarin; 95% confidence interviews shown in parentheses
^{b}Results based on S-6-hydroxywarfarin formation

### Discussion

This study expands the initial discovery that hydroxywarfarins inhibit CYP2C9 (18), and stresses the potential impact of hydroxywarfarins on metabolic capacity toward S-warfarin. These studies provide evidence that all hydroxywarfarins associate with CYP2C9 to block activity toward substrates. More importantly, hydroxywarfarins inhibited S-warfarin metabolism by CYP2C9 with both recombinant enzyme and human liver microsomes. As products of CYP2C9 and other CYPs, hydroxywarfarins are feedback inhibitors which suppress CYP2C9 activity toward warfarin and consequently link multiple CYP metabolic pathways to the overall efficiency of warfarin metabolism. These complex interactions could potentially lead to the biological accumulation of toxic levels of S-warfarin, the most active form of the drug (4). The use of racemic hydroxywarfarins, as opposed to pure enantiomers, in this study potentially underestimates the potency of these inhibitors, but provides upper bounds for the inhibition constants of the individual enantiomers.

The broad specificity of CYP2C9 for four of the racemic hydroxywarfarins indicates that these metabolites inhibit CYP2C9 metabolism of S-warfarin through cis and trans interactions (Scheme 1 below). 7-Hydroxywarfarin is the most abundant metabolite of warfarin in human urine and plasma (6, 8), and retains intermediate affinity for the enzyme, indicating that traditional product (cis) inhibition is possible. By contrast, 6-hydroxywarfarin did not significantly inhibit CYP2C9. The remaining hydroxywarfarins were effective trans-acting inhibitors on S-warfarin metabolism by CYP2C9. The most potent inhibitor in these studies was 10-hydroxywarfarin, which is the second most abundant metabolite in patient plasma (8). CYP3A4 generates this metabolite by metabolism of R-warfarin, thereby linking CYP3A4- and CYP2C9-mediated pathways to the overall efficiency of warfarin metabolism. Although 4′- and 8-hydroxywarfarin are usually minor plasma metabolites from CYP1A2 and CYP2C19 metabolism of R-warfarin (9, 10), these metabolites achieve high abundance in some patients (6, 8), and thus these metabolites could further contribute to the trans inhibition of S-warfarin metabolism by CYP2C9. For patients, the hydroxywarfarins may collectively compete for the CYP2C9 active site, thereby suppressing S-warfarin metabolism and potentiating supra-therapeutic dosing and possibly bleeding complications.

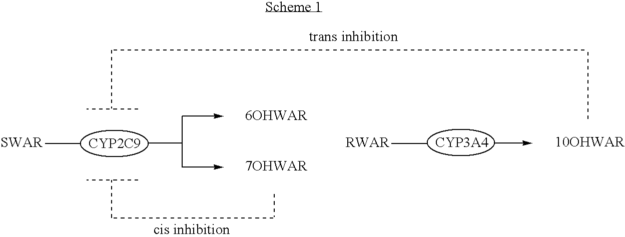

Feedback inhibition of CYP2C9 becomes physiologically relevant when hydroxywarfarins out compete S-warfarin. The degree of competition is a function of hydroxywarfarin concentration and binding affinity. Although the binding affinity of hydroxywarfarins for CYP2C9 is constant, the potential for in vivo inhibition of CYP2C9 is augmented by the differing plasma protein binding properties of warfarin and its metabolites. S-Warfarin has high affinity for plasma proteins (˜99% bound) (16), while 6-, 7-, and 8-hydroxywarfarin bind plasma proteins with three- to four-fold lower affinity (17). 4′-Hydroxywarfarin is thought to bind human plasma albumin with affinity comparable to warfarin (16), but the affinity of 10-hydroxywarfarin is unknown. The weaker affinity of hydroxywarfarin metabolites for plasma albumin increases their bioavailability, allowing hydroxywarfarins to potentially achieve higher unbound levels than warfarin. The level of CYP2C9 inhibition in vivo would therefore be a function of the ratio of S-warfarin to total hydroxywarfarin.

Locatelli et al (8) determined the levels of total hydroxywarfarins as well as S-warfarin in the plasma of long-term warfarin therapy patients. The levels of unbound hydroxywarfarin enantiomers have not been reported in human plasma, but the known affinities of warfarin and its metabolites for albumin (16, 17), and the Locatelli et al data to estimate the levels of unbound warfarin and hydroxywarfarins in plasma may be used. The unbound level of S-warfarin has been reported at 0.54% (25) which is in agreement with other similar reports (26). Therefore, the mean level of unbound S-warfarin in human plasma was estimated to be 2.7 ng/mL (8) the mean unbound concentration of 7- and 10-hydroxywarfarin was estimated to be 6.2 and 5.8 ng/mL respectively, given that ˜4% of the hydroxywarfarins are unbound (17). The inhibitory effect of each of these metabolites is expected to be additive, so 7-, and 10-hydroxywarfarin have a combined concentration approximately 4.5 times higher than S-warfarin. Both 6- and 8-hydroxywarfarin are significant metabolites in some patients (6, 8), and these metabolites would also contribute to inhibition of CYP2C9 activity, but no data were reported from those studies, so the unbound concentrations could not be estimated. These estimations are based on mean plasma values, but the extensive variation in individual patient levels for S-warfarin suggests that some populations are more prone to CYP2C9 inhibition by hydroxywarfarins while others may be resistant (8).

In addition to CYP activities toward R- and S-warfarin, the hydroxywarfarin levels may depend on the efficiency of elimination through excretion and further metabolism. Little is known about hydroxywarfarin transport. Nevertheless, patients with chronic kidney disease may have reduced ability to clear hydroxywarfarins, which would lead to product accumulation. Dreisbach et al. (27) observed a 50% increase in the S/R-warfarin ratio in end stage renal disease patients with CYP2C9*1/*1 (wild type) genotype. This observation was attributed to a selective decrease in hepatic CYP2C9 activity, albeit with an unknown mechanism. Limdi et al. (28) further demonstrated that such patients require significantly lower doses to maintain therapeutic anticoagulation, and are at increased risk for supratherapeutic dosing and major hemorrhage even after adjustment for CYP2C9 and vitamin K epoxide reductase (VKOR) genotype as well as other clinical factors. Feedback inhibition due to the accumulation of hydroxywarfarins would provide a mechanistic explanation for these observations. The processing of hydroxywarfarins into secondary metabolites such as glucuronides by UDP-glucuronosyltransferases (UGTs) would suppress the feedback inhibition and effectively create an extended metabolic pathway for warfarin. The potential significance of hydroxywarfarin conjugation by UGTs in human urine samples was demonstrated (6), and the enzymes responsible for those reactions identified (18). Although potentially significant (29), the combined effect of CYP and UGT activity on warfarin metabolism remains unresolved at this time.

For 8- and 4′-hydroxywarfarin, mixed inhibition toward CYP2C9 was observed, which could derive from two alternate mechanisms. The binding of S-warfarin to CYP2C9 may create a second binding site for other molecules as suggested by recent computational studies of the CYP2C9 active site (30). Hydroxywarfarins could bind to this secondary site and alter S-warfarin metabolism. Alternatively, the apparent mixed inhibition may be a consequence of the use of racemic hydroxywarfarin inhibitors. If the enantiomers of the hydroxywarfarins possess differing affinities toward CYP2C9, the resulting inhibition plot could appear biphasic and thus resemble mixed inhibition. Nevertheless, the current studies cannot distinguish between these possibilities. Further work is clearly needed to establish the mode of interaction between these molecules and CYP2C9.

CYP2C9 has long been recognized by metabolic and genetic studies as an important determinant of warfarin drug response (5, 31, 32). These studies provide a novel mechanistic explanation for reduced CYP2C9 metabolic capacity toward S-warfarin through a feedback inhibition mechanism. Hydroxywarfarins from multiple CYP pathways bind or re-bind CYP2C9, linking together the different oxidative pathways to the efficiency of S-warfarin metabolism (Scheme 2 below) and the potential toxic accumulation of the drug. The biological relevance of this mechanism requires further investigation to (1) establish the level of free metabolites driving inhibitory interactions, (2) determine the significance of conjugative reactions on hydroxywarfarin levels, and (3) characterize the enantiospecificity of interactions between CYPs and hydroxywarfarins.

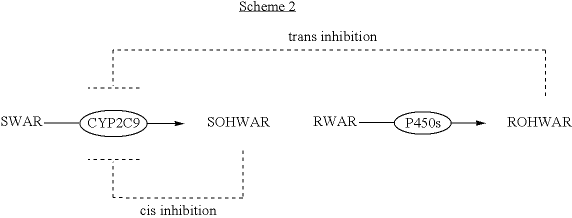

### REFERENCES FOR EXAMPLES 6

1. Greg C. Flaker and John Schutz. (2004) Why is Warfarin Underutilized in Patients with Atrial Fibrillation? Journal of Interventional Cardiac Electrophysiology 10, 21-25
2. Hirsh, J., Dalen, J. E., Anderson, D. R., Poller, L., Bussey, H., Ansell, J., and Deykin, D. (2001) Oral Anticoagulants: Mechanism of Action, Clinical Effectiveness, and Optimal Therapeutic Range. Chest 119, 8S-21 S
3. Kaminsky, L. S. and Zhang, Z. Y. (1997) Human P450 metabolism of warfarin. Pharmacology and Therapeutics 73, 67-74.
4. Eble, J. N., West, B. D., Link, K. P. (1966) A comparison of the isomers of warfarin. Biochemical Pharmacology 15, 1003-1006.
5. Rettie, A., Korzekwa, K R, Kunze, K L, Lawrence, R F, Eddy, A C, Aoyama, T, Gelboin, H V, Gonzalez, F J, Trager, W F (1992) Hydroxylation of warfarin by human cDNA-expressed cytochrome P-450: a role for P-4502C9 in the etiology of (S)-warfarin-drug interactions. Chem Res Toxicol. 5, 54-59.
6. Miller, G. P., Jones, D. R., Sullivan, S. Z., Mazur, A., Owen, S. N., Mitchell, N. C., Radominska-Pandya, A., and Moran, J. H. (2009) Assessing Cytochrome P450 and UDP-Glucuronosyltransferase Contributions to Warfarin Metabolism in Humans. Chem Res Tox. 22, 1239-45.
7. Steward, D. J., Haining, R. L., Henne, K. R., Davis, G., Rushmore, T. H., Trager, W. F., and Rettie, A. E. (1997) Genetic association between sensitivity to warfarin and expression of CYP2C9*3. Pharmacogenetics. 7, 361-367.
8. Locatelli, I., Kmetec, V., Mrhar, A., and Grabnar, I. (2005) Determination of warfarin enantiomers and hydroxylated metabolites in human blood plasma by liquid chromatography with achiral and chiral separation. Journal of Chromatography B. 818, 191-198.
9. Zhang, Z., Fasco, M. J., Huang, Z., Guengerich, F. P., and Kaminsky, L. S. (1995) Human cytochromes P450 1A1 and 1A2: R-warfarin as a probe. Drug Metab Dispos. 23, 1339-1345.
10. Kaminsky, L., de Morais, S., Faletto, M., Dunbar, D., and Goldstein, J. (1993) Correlation of human cytochrome P4502C substrate specificities with primary structure: warfarin as a probe. Mol Pharmacol. 43, 234-239.
11. Ngui, J. S., Chen, Q., Shou, M., Wang, R. W., Stearns, R. A., Baillie, T. A., and Tang, W. (2001) In Vitro Stimulation of Warfarin Metabolism by Quinidine: Increases in the Formation of 4′- and 10-Hydroxywarfarin. Drug Metab Dispos. 29, 877-886.
12. Holbrook, A. M., Pereira, J. A., Labiris, R., McDonald, H., Douketis, J. D., Crowther, M., and Wells, P. S. (2005) Systematic Overview of Warfarin and Its Drug and Food Interactions. Arch Intern Med. 165, 1095-1106.
13. Kunze, K. L., Eddy, A. C., Gibaldi, M., and Trager, W. F. (1991) Metabolic Enantiomeric Interactions: The Inhibition of Human (S)-Warfarin-7-Hydroxylase by (R)-Warfarin. Chirality. 3, 24-29.
14. Yamazaki, H., Shimada, T. (1997) Human liver cytochrome P450 enzymes involved in the 7-hydroxylation of R- and S-warfarin enantiomers. Biochem Pharmacol. 11, 1195-203.
15. Zhang, Z. Y., Kerr, J., Wexler, R. S., Li, H. Y. (1997) Warfarin Analog

Inhibition of Human CYP2C9-Catalyzed S-warfarin 7-Hydroxylation. Thrombosis Research. 88, 389-398.
16. O'Reilly, R. (1969) Interaction of the anticoagulant drug warfarin and its metabolites with human plasma albumin. J Clin Invest. 48, 193-202.
17. Chan, E., McLachlan, A J, Rowland, M. (1993) Warfarin metabolites: stereochemical aspects of protein binding and displacement by phenylbutazone. Chirality 5, 610-615.
18. Zielinska, A., Lichti, C. F., Bratton, S., Mitchell, N. C., Gallus-Zawada, A., Le, V.-H., Finel, M., Miller, G. P., Radominska-Pandya, A., and Moran, J. H. (2008) Glucuronidation of Monohydroxylated Warfarin Metabolites by Human Liver Microsomes and Human Recombinant UDP-Glucuronosyltransferases. J Pharmacol Exp Ther. 324, 139-148.
19. Wang, L., Christopher, L. J., Cui, D., Li, W., Iyer, R., Humphreys, W. G., and Zhang, D. (2008) Identification of the Human Enzymes Involved in the Oxidative Metabolism of Dasatinib: An Effective Approach for Determining Metabolite Formation Kinetics. Drug Metab Dispos. 36, 1828-1839.
20. Kuzmic, P. (1996) Program DYNAFIT for the analysis of enzyme kinetic data: application to HIV protease. Anal Biochem. 237: 260-273.
21. Burnham, K. B. and Anderson, D. R. (2002) Model Selection and Multimodel Inference: A Practical Information-Theoretic Approach. Springer-Verlag, New York, 2nd edition.
22. Takahashi H, Kashima T, Nomoto S, Iwade K, Tainaka H, Shimizu T, Nomizo Y, Muramoto N, Kimura S, Echizen H. (1998) Comparisons between in-vitro and in-vivo metabolism of (S)-warfarin: catalytic activities of cDNA-expressed CYP2C9, its Leu359 variant and their mixture versus unbound clearance in patients with the corresponding CYP2C9 genotypes. Pharmacogenetics. 5, 365-73.
23. Newton, D. J., Wang, R. W., Lu, A. Y. H. (1995) Evaluation of specificities in the in vitro metabolism of therapeutic agents by human liver microsomes. Drug Metabolism and Disposition. 23, 154-158.
24. L. L. Von Moltke et. al. (1997) Human cytochromes mediating N-demethylation of fluoxetine in vitro. Psychopharmacology. 132, 402-407.
25. Chan, E., McLachlan, A J, Pegg, M., MacKay, A D., Cole, R B., and Rowland, M. (1994) Disposition of warfarin enantiomers and metabolites in patients during multiple dosing with rac-warfarin. Br J clin Pharmac. 37, 563-569.
26. Banfield, C., O'Reilly, R., Chan, E., Rowland, M. (1983) Phenylbutazone-warfarin interaction in man: further stereochemical and metabolic considerations. Br. J. clin. Pharmac. 16, 669-675.
27. Dreisbach, A W., Japa, S., Gebrekal, A B., Mowry, S E., Lertora, J J L., Kamath, B L., Rettie, A E. (2003) Cytochrome P4502C9 activity in end-stage renal disease. Clin Pharm and Therapeutics. 73, 475-477.
28. Limdi, N A., Beasley, T M., Baird, M F., Goldstein, J A., McGwin, G., Arnett, D K., Acton, R T., and Allon, M. (2009) Kidney Function Influences Warfarin Responsiveness and Hemorrhagic Complications. J Am Soc Nephrol. 20, 912-921.
29. Jones, D. R., Moran, J. H., Miller, G. P. (2010) Warfarin and UDP-glucuronosyltransferases: writing a new chapter of metabolism. Drug Metabolism Reviews. 42, 53-59.
30. Clodfelter, K. H., Waxman, D. J., Vajda, S. (2006). Computational solvent mapping reveals the importance of local conformational changes for broad substrate specificity in mammalian cytochromes P450. Biochemistry 31, 9393-407.
31. Reynolds, K., Valdes, R J, Hartung, B R, Linder, M W (2007) Individualizing warfarin therapy. Personalized Medicine. 4, 11-31.
32. Au, N. and Rettie, A. E. (2008) Pharmacogenomics of 4-Hydroxycoumarin Anticoagulants. Drug Metab Rev. 40, 355-375.

What is claimed is:

1. A warfarin metabolic profile of a subject, the profile comprising the identity and stereochemistry of phase II warfarin metabolites in a sample from a subject, wherein the profile is stored in a computer readable form.

2. The metabolic profile of claim 1, further comprising the identity and stereochemistry of warfarin and phase I warfarin metaboles in a sample from the subject.

3. The metabolic profile of claims 1, wherein the profile further comprises the quantity of warfarin, phase I warfarin metabolites, and phase II warfarin metabolites in the sample.

4. The metabolic profile of claims 1, wherein the sample is a fluid sample or a tissue sample.

5. The metabolic profile of claims 1, wherein the sample is a fluid sample selected from the group comprising plasma and urine.

6. A method for determining a warfarin metabolic profile of a subject, the method comprising determining the identity and stereochemistry of phase II warfarin metabolites in a sample from the subject.

7. The method of claim 6, further comprising determining the identity and stereochemistry of warfarin and phase I warfarin metabolites in the sample.

8. The method of claim 7, wherein the identity of warfarin, phase I warfarin metabolites and phase II warfarin metabolites are determined simultaneously.

9. The method of claims 7, further comprising determining the quantity of warfarin, phase I warfarin metabolites and phase II warfarin metabolites in the sample.

10. The method of claims 6, wherein LC-MS/MS is used to determine the identity, stereochemistry, and quantity of warfarin, phase I warfarin metabolites, and phase II warfarin metabolites in the sample.

11. The method of claim 10, wherein the LC-MS/MS comprises two stages.

12. The method of claim 11, wherein the first phase resolves warfarin, phase I warfarin metabolites and phase II warfarin metabolites in the sample, and the second phase resolves the stereochemistry of the warfarin, phase I warfarin metabolites and phase II warfarin metabolites identified in the first phase.

13. The method of claims 11, wherein the first stage comprises, in part, reverse-phase HPLC.

14. The method of claims 11, wherein a gradient is used to resolve the stereoisomers in the second stage.

15. The method of claim 11, wherein the flow time for both stage one and stage two combined does not exceed about 25 min.

16. The method of claims 6, wherein the sample is a fluid sample or a tissue sample.

17. A method for determining an effective dose range of warfarin for a subject, the method comprising:
a. determining the warfarin metabolic profile of the subject;
b. comparing the profile to a database, wherein the database comprises at least one warfarin metabolic profile correlated to a known effective warfarin dose, and
c. selecting a database profile similar to the subject's warfarin metabolic profile, wherein the effective dose range of warfarin for the subject is the known effective warfarin dose correlated to the database profile.

18. The method of claim 17, wherein the warfarin metabolic profile of the subject is determined by:
a. administering warfarin to the subject,
b. collecting a sample from the subject,
c. and determining the identity and stereochemistry of phase II warfarin metabolites.

19. The method of claims 17, further comprising determining the identity and stereochemistry of warfarin and phase I warfarin metabolites in the sample.

20. The method of claims 17, further comprising determining the quantity of warfarin, phase I warfarin metabolites, and phase II warfarin metabolites in the sample.

21. The method of claims 17, wherein the sample is collected from the subject minutes after warfarin administration.

22. The method of claims 17, wherein the sample is collected from the subject hours after warfarin administration.

23. The method of claims 17, wherein the sample is collected from the subject before a difference in coagulation due to the warfarin administration is detectable.
